(19) United States
(12) Patent Application Publication
(10) Pub. No.: US 2026/0146071 A1
(43) Pub. Date: May 28, 2026

(54) CCR2 INHIBITORS AND METHODS OF USE

(71) Applicant: ORION BIOTECHNOLOGY USA Corp., Wilmington, DE (US)

(72) Inventors: Oliver Hartley, Troinex (CH); Irène Rossitto-Borlat, Carouge (CH); Fabrice Cerini, Puplinge (CH); Nicolas Calo, Gaillard (FR); Rachid Lani, Maxilly-sur-Léman (FR); Elsa Gonçalves Martins, Basel (CH); Ganesh Kumar Mothukuri, Geneva (CH); Marianne Paolini-Bertrand, Habère Poche (FR)

(73) Assignee: Orion Biotechnology USA Corp., Wilmington, DE (US)

(21) Appl. No.: 19/125,471

(22) PCT Filed: Oct. 31, 2023
(86) PCT No.: PCT/EP2023/080341

§ 371 Date: Apr. 29, 2025

Related U.S. Application Data
(60) Provisional application No. 63/420,780, filed on Oct. 31, 2022.

(51) Int. Cl. C07K 14/52; A61K 38/00; A61P 37/06

(52) U.S. Cl. CPC C07K 14/523; A61P 37/06; A61K 38/00; C07K 2319/00

(57) ABSTRACT

The present invention relates to CCR2 inhibitors that are variant CCL2 polypeptides comprising a variant N-terminal portion and a conserved C-terminal portion, and peptides and uses thereof.

### CROSS-REFERENCE TO RELATED APPLICATIONS

This application claims priority to U.S. provisional application No. 63/420,780 filed Oct. 31, 2022.

### FIELD OF THE INVENTION

The present invention relates to polypeptides, peptides, and conjugates with CCR2 inhibitory activity.

### BACKGROUND OF THE INVENTION

CCR2 is a receptor for the chemokine CCL2 (monocyte chemoattractant protein 1, MCP-1) and the closely related proteins CCL8 (MCP-2), CCL7 (MCP-3), and CCL13 (MCP-4). CCL2 binds preferentially to the receptor CCR2 and mediates cellular behaviors including monocyte chemotaxis. Studies have implicated CCL2-mediated monocyte infiltration in pain, cancer, and a range of inflammatory diseases.

Small molecule CCR2 inhibitors have been developed and tested for use in treating various diseases or disorders. The small molecule CCR2 inhibitors AZD2423, BMS-813160, CCX-140, and Cenicriviroc have been investigated for the treatment of diseases or disorders including posttraumatic neuralgia, neuropathic pain, inflammatory diseases, chronic obstructive pulmonary disease, diabetic polyneuropathy, cancer (including colorectal cancer, pancreatic cancer, pancreatic ductal adenocarcinoma, liver cancer, non-small cell lung cancer), diabetic nephropathy, diabetes, HIV infection, non-alcoholic steatohepatitis, liver fibrosis, liver cirrhosis, non-alcoholic fatty liver disease, or primary sclerosing cholangitis.

Given the potential applications for CCR2 inhibitors, there is a need for alternative CCR2 inhibitors with different pharmacological properties such as reduced toxicity and/or improved inhibitory potency.

### BRIEF SUMMARY OF THE INVENTION

The present invention provides polypeptides, peptides, and conjugates with CCR2 inhibitory activity.

In an embodiment of the present invention, there is provided a polypeptide comprising an N-terminal portion and a C-terminal portion, wherein the N-terminal portion comprises an amino acid sequence with 0, 1, 2, 3, or 4 amino acid substitutions relative to any one of SEQ ID NO: 40-70 and 76-145, and wherein the C-terminal portion comprises an amino acid sequence at least 70% identical to SEQ ID NO: 71, 72, 74, or 75.

In an embodiment of the present invention, there is provided a peptide comprising an amino acid sequence with 0, 1, 2, 3, or 4 amino acid substitutions relative to any one of SEQ ID NO: 40-70 and 76-145.

In an embodiment of the present invention, there is provided a nucleic acid molecule encoding the polypeptide or the peptide as described herein.

In an embodiment of the present invention, there is provided a vector comprising the nucleic acid molecule as described herein.

In an embodiment of the present invention, there is provided a host cell comprising the nucleic acid molecule or the vector as described herein.

In an embodiment of the present invention, there is provided a pharmaceutical composition comprising the polypeptide, the peptide, the nucleic acid molecule, or the vector as described herein, and a pharmaceutically acceptable carrier.

In an embodiment of the present invention, there is provided a method of inhibiting CCR2 signaling in a cell, comprising contacting the cell with the polypeptide, the peptide, the nucleic acid molecule, the vector, or the pharmaceutical composition as described herein.

In an embodiment of the present invention, there is provided a method of treating or preventing a disease or disorder associated with CCR2 signaling in a subject, comprising administering to the subject the polypeptide, the peptide, the nucleic acid molecule, the vector, or the pharmaceutical composition as described herein.

In an embodiment of the present invention, there is provided use of the polypeptide, the peptide, the nucleic acid molecule, the vector, or the pharmaceutical composition as described herein for inhibiting CCR2 signaling in a cell.

In an embodiment of the present invention, there is provided use of the polypeptide, the peptide, the nucleic acid molecule, the vector, or the pharmaceutical composition as described herein for treating or preventing a disease or disorder associated with CCR2 signaling in a subject.

The advantages and features of the present invention will become better understood with reference to the following more detailed description and claims taken in conjunction with the accompanying drawings, tables and sequence listings.

### BRIEF DESCRIPTION OF THE DRAWINGS

Embodiments will be described, by way of example only, with reference to the accompanying figures.

FIG. 1 illustrates the HPLC profile of purified 1P2-CCL2 (Met64Nle OB-004).

FIG. 2 illustrates the MALDI-TOF/TOF spectrum of purified 1P2-CCL2 (Met64Nle OB-004).

FIG. 3 illustrates dose-inhibition curves obtained for 1P2-CCL2 (Met64Nle OB-004) in four independent experiments.

FIG. 4 illustrates results of an example experiment of a CCR2 inhibitory potency assay, testing four known small molecule CCR2 inhibitors and 1P2-CCL2 (Met64Nle OB-004).

FIG. 5 illustrates compiled data from 4 experiments relating to CCR2 inhibitory potency assays, testing four known small molecule CCR2 inhibitors and 1P2-CCL2 (Met64Nle OB-004).

FIG. 6 illustrates number of monocytes (D1) captured/transmigrated on acute activated HUVECS after 4 hours stimulation with TNF-alpha in the presence of 1P2-CCL2 (Met64Nle OB-004) and GT-73.

FIG. 7 illustrates number of monocytes (D1) captured/transmigrated on acute activated HUVECS after 18 hours stimulation with TNF-alpha in the presence of 1P2-CCL2 (Met64Nle OB-004) and GT-73.

FIG. 8 illustrates number of monocytes (D2) captured/transmigrated on acute activated HUVECS after 4 hours stimulation with TNF-alpha in the presence of 1P2-CCL2 (Met64Nle OB-004) and GT-73.

FIG. 9 illustrates number of monocytes (D2) captured/transmigrated on acute activated HUVECS after 18 hours stimulation with TNF-alpha in the presence of 1P2-CCL2 (Met64Nle OB-004) and GT-73.

FIG. 10 illustrates number of monocytes (D3) captured/transmigrated on acute activated HUVECS after 4 hours stimulation with TNF-alpha in the presence of 1P2-CCL2 (Met64Nle OB-004) and GT-73.

FIG. 11 illustrates number of monocytes (D3) captured/transmigrated on acute activated HUVECS after 18 hours stimulation with TNF-alpha in the presence of 1P2-CCL2 (Met64Nle OB-004) and GT-73.

FIG. 12 illustrates number of monocytes (D4) captured/transmigrated on acute activated HUVECS after 4 hours stimulation with TNF-alpha in the presence of 1P2-CCL2 (Met64Nle OB-004) and GT-73.

FIG. 13 illustrates number of monocytes (D4) captured/transmigrated on acute activated HUVECS after 18 hours stimulation with TNF-alpha in the presence of 1P2-CCL2 (Met64Nle OB-004) and GT-73.

FIG. 14 illustrates number of monocytes (D1-4) captured/transmigrated on acute activated HUVECS after 4 and 18 hours stimulation with TNF-alpha in the presence of 1P2-CCL2 (Met64Nle OB-004) and GT-73.

FIG. 15 illustrates number of monocytes (D1-4) captured/transmigrated on acute activated HUVECS after 4 hours stimulation with TNF-alpha in the presence of 1P2-CCL2 (Met64Nle OB-004) and GT-73.

FIG. 16 illustrates number of monocytes (D1-4) captured/transmigrated on acute activated HUVECS after 18 hours stimulation with TNF-alpha in the presence of 1P2-CCL2 (Met64Nle OB-004) and GT-73.

FIG. 17 illustrates number of monocytes (D1-4) captured/transmigrated on acute activated HUVECS after 4 and 18 hours stimulation with TNF-alpha in the presence of 1P2-CCL2 (Met64Nle OB-004) and GT-73.

FIG. 18 illustrates 1P2-CCL2 (Met64Nle OB-004) potently blocks monocyte transmigration in HUVECS activated with TNF-alpha for 18 hours.

FIG. 19 illustrates CCR2 inhibition with chimeric murine CCL2 variants 1P2 (segmented line) and 1P8 (solid line).

FIG. 20 illustrates in vivo inhibition of monocyte/macrophage recruitment to the peritoneal cavity with murine chimeric CCL2 variant 1P8 polypeptide (c1P8-CCL2).

FIG. 21 illustrates body weight following control (sham) treatment, treatment with bleomycin only, or treatment with bleomycin and the CCR2 inhibitor. Measured as a percent of the total body weight at Day −2 of the study.

FIG. 22 Analysis of lung infiltrating macrophages by flow cytometry. Total CD64+ macrophages (As a % of cells FIG. 22A or as number of cells FIG. 22B), CD64+ Ly6C−/low SiglecF− interstitial macrophages (As a % of cells FIG. 22C or as number of cells FIG. 22D), and CD64+ Ly6C+ SiglecF− monocyte-derived macrophages (As a % of cells FIG. 22E or as number of cells FIG. 22F). Only comparison of all groups with vehicle group; homogeneity of variances (Bartlett's test); for homogenous variances, one-way ANOVA test followed by Dunnett's post hoc test; for heterogenous variances, Kruskal-Wallis test followed by Dunn's post-hoc test; data are represented as mean+/−SEM.

### DETAILED DESCRIPTION

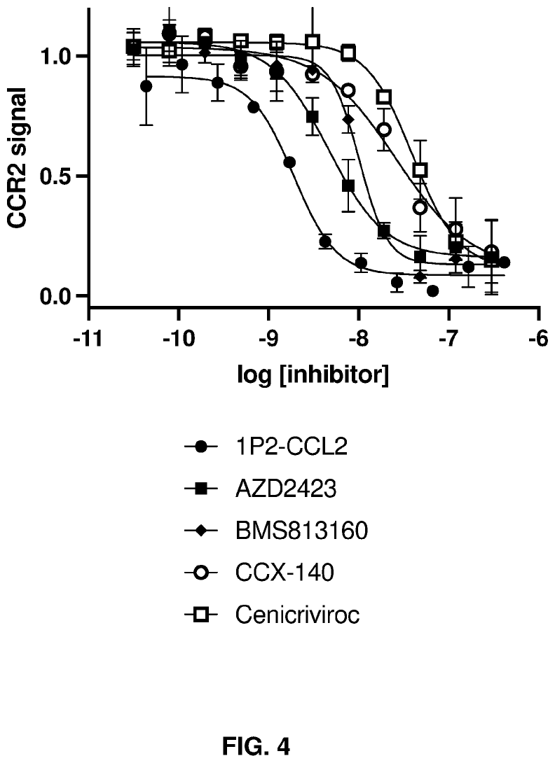

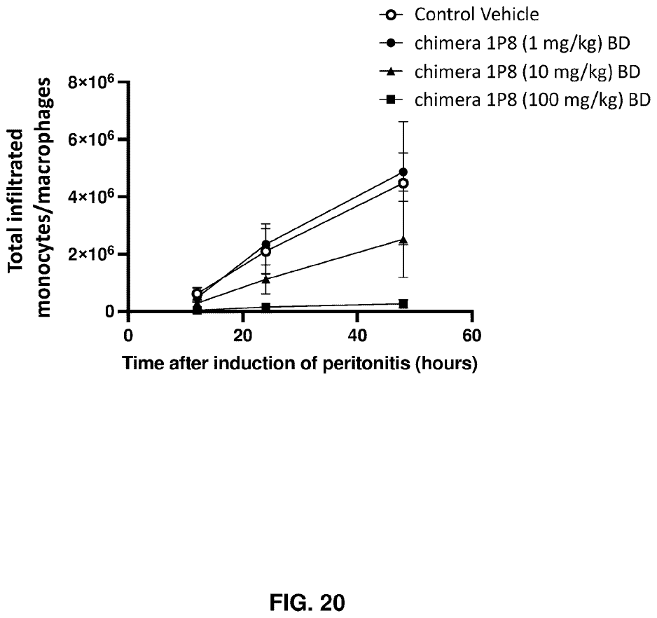

The present inventors have discovered peptides that can be used to create variants of CCL2 with CCR2-inhibitory activity. The peptides may be used to replace the native N-terminal portion of CCL2 or a CCL2 analog to produce a CCL2 variant polypeptide with CCR2-inhibitory activity. As demonstrated herein by way of example, a CCL2 variant polypeptide, wherein the peptide FTNPTWAPVT (SEQ ID NO: 40) replaces the first ten N-terminal residues of a human CCL2, exhibits 3.5 to 36.4 fold greater potency in CCR2 inhibition compared to the small molecule CCR2 inhibitors AZD2423, BMS-813160, CCX-140, and Cenicriviroc as measured by IC₅₀ (FIG. 4). In another example, a chimeric CCL2 variant comprising the N-terminal portion AFSIMQAPVT (SEQ ID NO: 46) and a C-terminal portion from mouse CCL2 (SEQ ID NO: 72), effectively inhibits the recruitment of monocytes and macrophages to the peritoneal cavity during peritonitis (FIG. 20). Other peptides providing CCR2-inhibitory activity are described herein.

Sequences, compositions, and methods for carrying out the invention are presented in terms of examples and embodiments in the present disclosure. However, the invention is not limited to the described examples and embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

### Molecules of the Invention

Peptides providing CCR2-inhibitory activity and which may be incorporated into polypeptides, such as CCL2 variants, and into conjugates are provided.

In some embodiments, there is provided a polypeptide comprising an N-terminal portion and a C-terminal portion, wherein the polypeptide is a CCL2 variant and is a CCR2 inhibitor. As used herein, a “CCL2 variant”, “CCL2 variant polypeptide”, “CCL2 derivative”, or “CCL2 derivative polypeptide” according to the present invention refers to a polypeptide derived from CCL2, for example human CCL2 (SEQ ID NO: 1) or a mouse CCL2 (SEQ ID NO: 73), in which the N-terminal portion comprises a peptide of the invention as described herein and the C-terminal portion comprises an amino acid sequence at least 70%, at least 75%, at least 80%, at least 85%, at least 90%, at least 95%, at least 99%, at least 99.9%, or 100% identical to SEQ ID NO: 71, 72, 74, or 75. In some embodiments, the N-terminal portion comprises or consists of an amino acid sequence with 0, 1, 2, 3, or 4 amino acid substitutions relative to any one of SEQ ID NO: 9-70 and 76-145. In some embodiments, the N-terminal portion comprises or consists of the amino acid sequence of any one of SEQ ID NO: 146-149.

As described herein, methionine residues can be conservatively substituted with non-oxidizable amino acid analogs or amino acid derivatives to reduce complications from methionine oxidation during synthesis. In some embodiments, one or more methionine residues in the polypeptides, peptides, and conjugates of the present invention are conservatively substituted with an amino acid analog or an amino acid derivative such as, but not limited to, norleucine (Nle). In some embodiments, the C-terminal portion of the CCL2 variant comprises a norleucine (Nle) residue at position 54 relative to SEQ ID NO: 74 (position 64 relative to SEQ ID NO: 1). In some embodiments, the C-terminal portion of the CCL2 variant comprises a norleucine (Nle) residue at one or more of positions 9, 12, and 62 relative to SEQ ID NO: 72.

As used herein, the term “CCR2 inhibitor” and “CCR2 antagonist” may be used interchangeably to mean a polypeptide, peptide, conjugate, small molecule, or other compound that inhibits one or more biological and/or pathological activities induced by the agonism, activation, or signaling of CCR2. CCR2 inhibitors may prevent the binding of ligands or pathogens by achieving partial or complete occupation of the site or sites on CCR2 that they require for interaction (orthosteric inhibitors). For example, a CCR2 inhibitor may block the binding of native CCL2 ligand to CCR2, preventing normal activation of CCR2, or block the binding of a synthetic CCR2 agonist to CCR2. Alternatively, CCR2 inhibitors may prevent the binding of ligands or pathogens by engaging sites on CCR2 and inducing CCR2 to adopt a conformation or conformations that cannot be recognized by the ligands or pathogens (allosteric inhibitors). CCR2 inhibitors may inhibit the entire repertoire or only a subset of CCR2 intracellular signaling pathways and pathogenic interactions.

1P2-CCL2 (OB-004) is an exemplary CCL2 variant with improved CCR2 inhibitory potency comprising the peptide FTNPTWAPVT (SEQ ID NO: 40) discovered by the present inventors in a phage display campaign (the general method outlined in Gaertner et al. PNAS, 2008, 105(46):17706-17711) using a library consisting of CCL2 variants in which the first six N-terminal residues were randomized (all possible amino acids except cysteine). At the end of phage library selection performed on CHO-CCR2 cells, sequencing revealed 1P2-CCL2 (OB-004) to be the second-most abundant clone. The N-terminal sequence of 1P2-CCL2 (OB-004) (residues 1-10) was FTNPTWAPVT (SEQ ID NO: 40). A panel of CCL2 variants, including 1P2-CCL2 (OB-004), was isolated by phage display and produced using a multiplex synthesis approach (as according to the general method outlined in (Paolini et al. JBC, 2018, 293(49):19092-19100), with a C-terminal core fragment in which methionine residue 64 of CCL2 (residue 54 of SEQ ID NO: 74 or residue 64 of SEQ ID NO: 1) was substituted with the isosteric but non-oxidizable non-natural amino acid norleucine (Nle) in order to reduce complications from methionine oxidation during synthesis. Experiments revealed 1P2-CCL2 (Met64Nle OB-004) and a number of other CCR2 variants to inhibit CCR2 signaling activity by the native ligand, CCL2.

In an embodiment, the polypeptide of the present invention comprises an amino acid sequence with 0, 1, 2, 3 or 4 amino acid substitutions relative to any one of SEQ ID NO: 9-70 and 76-145. In some embodiments, the polypeptide of the present invention comprises the amino acid sequence of any one of claims 146-149. In some embodiments, In some embodiments, this amino acid sequence is located near the N terminus of the polypeptide. In some embodiments, this amino acid sequence is located such that the beginning of the amino acid sequence lies within 15 residues of the N terminus of the polypeptide, such as within 15, 12, 10, 8, 6, 5, 4, 3, 2, 1 residues of the N terminus, or consists of the N terminus of the polypeptide.

In some embodiments, the polypeptide according to the present invention comprises an N-terminal portion and a C terminal portion, wherein said N-terminal portion comprises an amino acid sequence with 0, 1, 2, 3, or 4 amino acid substitutions relative to any one of SEQ ID NO: 9-70 and 76-145 or comprises the amino acid sequence of any one of SEQ ID NO: 146-149, and the C-terminal portion comprises an amino acid sequence at least 70% identical to SEQ ID NO: 71, 72, 74, or 75. In some embodiments, the N-terminal portion comprises the amino acid sequence FTNPTWXXXX (SEQ ID NO: 146), wherein X is any amino acid, and the C-terminal portion comprises an amino acid sequence at least 70% identical to SEQ ID NO: 71, 72, 74, or 75. In some embodiments, the N-terminal portion comprises the amino acid sequence FTNPTW[A or D or R or S or K or Q][P or A or T or G or S or Q or R or H or E][V or F or Q or G or S or H or L or Y][T or V or Q or S or A] (SEQ ID NO: 147) and the C-terminal portion comprises an amino acid sequence at least 70% identical to SEQ ID NO: 71, 72, 74, or 75. In some embodiments, the N-terminal portion comprises the amino acid sequence of any one of SEQ ID NO: 40 and 76-99 and the C-terminal portion comprises an amino acid sequence at least 70% identical to SEQ ID NO: 71, 72, 74, or 75. In some embodiments, the N-terminal portion comprises the amino acid sequence FPX₁DGWX₂X₃X₄X₅(SEQ ID NO: 148), wherein X₁ is Methionine or Norleucine (Nle), and X₂-X₅ is any amino acid, and the C-terminal portion comprises an amino acid sequence at least 70% identical to SEQ ID NO: 71, 72, 74, or 75. In some embodiments, the N-terminal portion comprises the amino acid sequence FPX₁DGW[A or R or G or H or V or Q][P or S or G or E][V or R or L or E or T or G or Q][T or V or Q] (SEQ ID NO: 149), wherein X₁ is Methionine or Norleucine (Nle), and the C-terminal portion comprises an amino acid sequence at least 70% identical to SEQ ID NO: 71, 72, 74, or 75. In some embodiments, the N-terminal portion comprises the amino acid sequence of any one of SEQ ID NO: 41 and 100-145 and the C-terminal portion comprises an amino acid sequence at least 70% identical to SEQ ID NO: 71, 72, 74, or 75.

In some embodiments of polypeptides of the present invention, the N-terminal portion consists of no more than 12 amino acids, such as no more than 12, 11, 10, 9, 8, 7, 6, 5, or 4 amino acids. In some embodiments, the N-terminal portion consists of 11 amino acids, consists of 10 amino acids, or consists of 8 amino acids. In an embodiment, the N-terminus of the C-terminal portion adjoins directly to the C-terminus of the N-terminal portion, i.e. the N-terminal portion and the C terminal portion are directly adjoined. In some embodiments, the N-terminal portion is adjoined to the C-terminal portion via a peptide linker. In some embodiments, the N-terminal portion is located at the extreme N terminus of the polypeptide.

In some embodiments, there is provided a peptide comprising an amino acid sequence with 0, 1, 2, 3, or 4 amino acid substitutions relative to any one of SEQ ID NO: 9-70 and 76-145. In some embodiments, there is provided a peptide comprising an amino acid sequence with 0, 1, 2, 3, or 4 amino acid substitutions relative to any one of SEQ ID NO: 40-70 and 76-145. In some embodiments, there is provided a peptide comprising the amino acid sequence of any one of SEQ ID NO: 146-149. In some embodiments, there is provided a peptide comprising the amino acid sequence FTNPTWXXXX (SEQ ID NO: 146), wherein X is any amino acid. In some embodiments, there is provided a peptide comprising the amino acid sequence FTNPTW[A or D or R or S or K or Q][P or A or T or G or S or Q or R or H or E][V or F or Q or G or S or H or L or Y][T or V or Q or S or A] (SEQ ID NO: 147). In some embodiments, there is provided a peptide comprising the amino acid sequence of any one of SEQ ID NO: 40 and 76-99. In some embodiments, there is provided a peptide comprising the amino acid sequence FPX₁DGWX₂X₃X₄X₅(SEQ ID NO: 148), wherein X₁ is Methionine or Norleucine (Nle), and X₂-X₅ is any amino acid. In some embodiments, there is provided a peptide comprising the amino acid sequence FPX₁DGW[A or R or G or H or V or Q][P or S or G or E][V or R or L or E or T or G or Q][T or V or Q] (SEQ ID NO: 149), wherein X₁ is Methionine or Norleucine (Nle). In some embodiments, there is provided a peptide comprising the amino acid sequence of any one of SEQ ID NO: 41 and 100-145.

In some embodiments, there is provided a conjugate comprising the polypeptide, such as a CCL2 variant polypeptide, or the peptide as described herein conjugated to a moiety. As used in reference to a conjugate, the term “moiety” refers to an atom, molecule, or compound that is conjugated to the polypeptide or the peptide of the present invention. In some embodiments, conjugation of the moiety to the polypeptide or peptide allows delivery of the moiety to CCR2-expressing cells, tissues, and/or tumors without inducing CCR2 signaling. In some embodiments, the moiety is an immunoglobulin domain such as an Fc domain of an IgG, IgM, IgE, IgD, or IgA heavy chain, or an Fc domain of a lambda or kappa light chain. In some embodiments, the moiety is a chemokine domain, such as a domain of a mouse CCL2 or a human CCL2. In some embodiments, the moiety is a toxin such as a toxin that enables the killing of a CCR2-expressing cell or the killing of a pathogen inside a CCR2-expressing cell. In some embodiments, the toxin is a cytotoxic agent, an anti-tumor agent, a chemotherapeutic agent, an antiviral agent, or an antibacterial agent. In some embodiments, the toxin is pseudomonas exotoxin A, diptheria toxin, a ribosome inactivating protein, or saporin. In some embodiments, the moiety is a carrier protein such as albumin. In some embodiments, the moiety is a polymer such as polyethylene glycol. In some embodiments, the moiety is a lipid. In some embodiments, the moiety is a detectable marker such as a fluorescent molecule (e.g. green fluorescent protein, red fluorescent protein, yellow fluorescent protein, or an AlexaFluor™), a dye, or a radioisotope. The moiety may be conjugated to the polypeptide or peptide by a variety of means known in the art. By “conjugated”, it is meant that the polypeptide or peptide of the invention is associated with the moiety by a covalent or non-covalent bond. In embodiments wherein the moiety is an amino acid polymer, then the polypeptide or peptide of the invention may be conjugated to the moiety by a peptide bond, optionally through a linker amino acid sequence and/or through a disulfide bond.

The present invention provides polypeptides, peptides, and conjugates as disclosed above and, moreover, nucleic acid molecules encoding said polypeptides, peptides and conjugates. In some embodiments, the nucleic acid molecules encoding polypeptides, peptides, and/or conjugates of the present invention are RNA or DNA. The skilled person is able to design or identify nucleic acid molecules encoding said polypeptides, peptides, and conjugates of the present invention using methods known in the art. In some embodiments, the nucleic acid molecules encoding polypeptides, peptides, and/or conjugates of the present invention are incorporated into a vector, such as a plasmid, episome, artificial chromosome, virus, or a viral vector. In some embodiments, the nucleic acid molecules encoding polypeptides, peptides, and/or conjugates of the present invention or the vector comprising said nucleic acid molecule are comprised within a host cell to enable expression of the polypeptides, peptides, and/or conjugates of the present invention. In some embodiments, the host cell is a bacterial cell, a yeast cell, a vertebrate cell, a mammalian cell, a human cell, or a cell of an immortalized cell line such as a CHO cell, a HEK cell, or a HeLa cell.

Polypeptides and peptides are polymers that comprise amino acids linked by peptide bonds. As used herein, the term “amino acid” is used to describe any amino acid, natural or otherwise, that can be incorporated into a polypeptide or a peptide. Amino acids are small molecules comprising an amine (—NH₂) group, a carboxyl (—COOH), and a variable side chain (R-group) specific to each amino acid. Amino acids are covalently linked by peptide bonds between the amine group of one amino acid to the carboxyl group of another amino acid to form polypeptides. Amino acids within a polypeptide are often referred to in the art as “residues”.

Polypeptides and peptides may comprise post-translational modifications such as, for example, phosphorylation, glycosylation, ubiquitination, nitrosylation, methylation, acetylation, lipidation, acylation, prenylation, alkylation, oxidation, or other modifications known in the art.

Polypeptides and peptides may comprise amino acid analogs. As used herein, the term “amino acid analogs” describes artificial, synthetic, or unnatural amino acids beyond the 20 genetically-encoded amino acids, such as for example the amino acid analogs described in Zou et al. (2018, Biotechnology Advances 36(7), 1917-1927). Examples of amino acid analogs that can be incorporated into polypeptides, peptides, and conjugates of the present invention include, but are not limited to, norleucine (Nle), β-amino acids, homo-amino acids, synthetic proline and pyruvic acid derivatives, 3-substituted alanine derivatives, glycine derivatives, ring-substituted phenylalanine and tyrosine derivatives, linear core amino acids, N-methyl amino acids, and amino acids with synthetic R-groups. Polypeptides and peptides may also comprise amino acid derivatives. As used herein, the term “amino acid derivatives” describes amino acids that have been derived from the modification of one of the 20 genetically-encoded amino acids. Amino acid derivatives can be synthetic, e.g. made in vitro by chemical reaction, or they can be naturally occurring in organisms, e.g. in vivo metabolites. An example of an amino acid derivative is pyroglutamate/pyroglutamic acid, a cyclized derivative of glutamine in which the free amino group of glutamic acid cyclizes to form a lactam.

The potency of inhibitory molecules is sometimes measured and expressed in the art in terms of “IC₅₀” values obtained from an inhibitory potency assay. Numerous inhibitory potency assays are known in the art and may be used to measure the IC₅₀ value of the present invention, such as for example an assay measuring competition with a labelled tracer molecule for binding to the receptor of interest or measuring the reduction in signaling induced by the natural agonist. The IC₅₀ is typically defined as the concentration of the agent at which 50% of a signal elicited via the receptor by a natural agonist is inhibited by the agent. IC₅₀ may sometimes be reported as “pIC₅₀”, which is the negative log of the IC₅₀ value in moles per liter (molar or M).

In some embodiments, the polypeptide, the peptide, or the conjugate of the present invention inhibits CCR2 with an IC₅₀ of less than 300 nM, less than 200 nM, less than 100 nM, less than 50 nM, less than 30 nM, less than 10 nM, or less than 3 nM.

In some embodiments, the polypeptides of the present invention are related to or derived from CCL2. In some embodiments, the CCL2 is human CCL2 (SEQ ID NO: 1), mouse CCL2 (SEQ ID NO: 73), a portion of a human CCL2 (SEQ ID NO: 2-5 and 74-75), or a portion of a mouse CCL2 (SEQ ID NO: 71 or 72). The polypeptides may comprise the sequence of SEQ ID NO: 2, 3, 4, 5, 71, 72, 74, or 75 or a variant, homologue (orthologue, allelic variant, derivative, functional mutant) or fragment thereof.

According to the present invention, a sequence is said to bear similarity to, or to be a homologue of, SEQ ID NO: 2, 3, 4, 5, 71, 72, 74, or 75 if said sequence is more than 70% identical to SEQ ID NO: 2, 3, 4, 5, 71, 72, 74, or 75. In some embodiments, said sequence is a C-terminal portion having more than 70%, more than 75%, more than 80%, more than 85%, more than 90%, more than 95%, more than 99%, or 99.9% sequence identity to SEQ ID NO: 2, 3, 4, 5, 71, 72, 74, or 75. The terms “identity” or “similarity” refers to sequence similarity between two polypeptides. Identity can be determined by comparing each position in the aligned sequences. A degree of identity between amino acid sequences is a function of the number of identical or matching amino acids at positions shared by the sequences, for example, over a specified region. Optimal alignment of sequences for comparisons of identity may be conducted using a variety of algorithms, as are known in the art, including the ClustalW program, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described in Altschul et al., 1990, J. Mol. Biol. 215:403-10 (using the published default settings). For example, the “BLAST 2 Sequences” tool, available through the National Center for Biotechnology Information may be used, selecting the “blastp” program at the following default settings: expect threshold 10; word size 3; matrix BLOSUM 62; gap costs existence 11, extension 1. In another embodiment, the person skilled in the art can readily and properly align any given sequence and deduce sequence identity, similarity, and/or homology by mere visual inspection.

A sequence is also said to bear similarity to or to be a homologue of SEQ ID NO: 2, 3, 4, 5, 71, 72, 74, or 75 if it contains one or more conservative substitutions with respect to SEQ ID NO: 2, 3, 4, 5, 71, 72, 74, or 75. Conservative substitutions are substitutions in the sequence of a peptide or polypeptide that do not lead to a significant loss of function or which lead only to a small loss of function. Such a loss of function due to one or more conservative substitutions may be considered not to be significant if said loss amounts to less than 20%, less than 15%, less than 10%, less than 6%, or less than 4% with respect to the function of the polypeptide having the unsubstituted sequence. Conservative substitutions are often substitutions wherein an amino acid side chain is replaced by an amino acid side chain that is related, or similar in physicochemical properties, to the replaced residue. Such conservative substitutions may be made, for example, using one of the 20 natural amino acids according to Table 2 wherein amino acids in the same block in the middle column and preferably in the same line in the right-hand column may be substituted for each other. Conservative substitutions may also be made using amino acid analogs or amino acid derivatives such as, for example, a substitution of methionine with norleucine (Nle).

TABLE 2

| | | |
|---|---|---|
| aliphatic | Non-polar | G A P |
| | | I L V |
| | polar uncharged | C S T M |
| | | N Q |
| | polar charged | D E |
| | | K R H |
| aromatic | | H F W Y |

According to the present invention, a sequence is said to bear similarity to, or to be a homologue of, SEQ ID NO: 2, 3, 4, 5, 71, 72, 74, or 75 if more than 30% of residues in said sequence are identical or conservatively substituted with respect to SEQ ID NO: 2, 3, 4, 5, 71, 72, 74, or 75. In some embodiments, more than 35%, 40%, 45%, 50%, 55%, 60%, 65%, 70%, 75%, 80%, 85%, 90%, 95%, 98% or 99% of said sequence are identical or conservatively substituted with respect to SEQ ID NO: 2, 3, 4, 5, 71, 72, 74, or 75.

In some embodiments, the invention provides polypeptides comprising fragments of SEQ ID NO: 2, 3, 4, 5, 71, 72, 74, or 75. The fragments should comprise at least ‘n’ consecutive amino acids from the SEQ ID NO: 2, 3, 4, 5, 71, 72, 74, or 75 and, depending on the particular sequence, ‘n’ is 5 or more (such as more than 6, 8, 10, 12, 14, 16, 18, 20, 22, 24, 26, 28, 3′0, 32, 34, 36, 38, 40, 42, 44, 46, 48, 50, 52, 54, 55, 56, or 57).

In some embodiments, the present invention provides a polypeptide comprising an N-terminal portion and a C-terminal portion, wherein the N-terminal portion comprises an amino acid sequence with 0, 1, 2, 3, or 4 amino acid substitutions relative to any one of SEQ ID NO: 9-70 and 76-145 and the C-terminal portion comprises an amino acid sequence at least 70% identical to SEQ ID NO: 2, 3, 4, 5, 71, 72, 74, or 75. In some embodiments, the present invention provides a peptide comprising an amino acid sequence with 0, 1, 2, 3, or 4 amino acid substitutions relative to any one of SEQ ID NO: 9-70 and 76-145. In some embodiments of the above polypeptide or peptide, the substitution relative to any one of SEQ ID NO: 9-70 and 76-145 is a conservative amino acid substitution as known in the art or as defined herein.

### Preparation of Polypeptides, Peptides, and Conjugates

Polypeptides and peptides of the present invention, isolated or for inclusion in a conjugate, can be prepared in many ways, for example, using known techniques of protein chemistry (for example, chemical peptide synthesis) or molecular biology (i.e. genetic engineering and fermentation—in general, biotechnology).

The polypeptides, peptides, and conjugates of the present invention may be prepared using the known techniques of protein chemistry as described, for example, in Gaertner et al., (PNAS, 2008, 105(46):17706-17711).

A method of preparing polypeptides, peptides, and conjugates of the present invention involves in vitro chemical synthesis. The polypeptides, peptides, and conjugates may be synthesized in part or in whole using chemical means. For example, solid-phase peptide synthesis, such as methods based on tBoc or Fmoc chemistry, may be used. Enzymatic synthesis may also be used in part or in full.

In addition, the polypeptides, peptides, and conjugates of the present invention can be prepared using genetic engineering. The peptides, polypeptides, and conjugates of the present invention may be produced by culturing a host cell comprising a nucleic acid molecule expressing the peptides, polypeptides, or conjugates (or the amino acid portions of the conjugates) of the present invention under conditions which induce expression of the peptides, polypeptides, or conjugates. In some embodiments, the host cell is a bacterial cell (e.g. E. coli), a yeast cell (e.g. Saccharomyces cerevisiae), or a mammalian cell (e.g. a human cell, a mouse cell, a CHO cell, a HEK cell, a HeLa cell).

Biological synthesis other than by expression in a host cell may be used, e.g. the polypeptides, peptides, or conjugates (or the amino acid portions of the conjugates) of the present invention may be produced by translation from RNA in vitro. Polypeptides, peptides, or conjugates (or the amino acid portions of the conjugates) of the present invention can, for example, also be prepared by digesting longer polypeptides using proteases.

Biological methods, including genetic engineering, fermentation, and expression are in general restricted to the production of polypeptides based on L-amino acids, but manipulation of translation machinery in vivo or in vitro (e.g. of aminoacyl tRNA molecules) can be used to allow the introduction of D-amino acids (or of other non-natural amino acids, such as iodotyrosine or methylphenylalanine, azidohomoalanine, etc.). Where D-amino acids are included, however, it is preferred to use chemical synthesis. Polypeptides, peptides, or conjugates of the present invention may have covalent modifications at the C-terminus and/or N-terminus.

### Pharmaceutical Compositions

The present invention provides pharmaceutical compositions comprising polypeptides, peptides, conjugates, or a nucleic acid or vector encoding said polypeptides, peptides, or conjugates according to the present invention, and a pharmaceutically acceptable carrier, excipient, and/or stabilizer. The pharmaceutical compositions of the present invention may be provided for use as a medicament. Pharmaceutical compositions according to the present invention may comprise any embodiment of the present invention, i.e., a polypeptide, peptide, conjugate, or a nucleic acid or vector encoding said polypeptide, peptide, or conjugate according to the present invention. The preparation of pharmaceutical compositions is well known to the person skilled in the art.

A pharmaceutical composition of the present invention may be administered to a subject in a therapeutically effective amount. As used herein, a “therapeutically effective amount” means an amount of the composition or therapeutic agent effective to provide a therapeutic, prophylactic or diagnostic benefit to a subject. In some embodiments, a therapeutically effective amount of the composition is an amount capable of inducing a clinical response in a subject in the treatment of a particular disease or disorder. Determination of a therapeutically effective amount of the composition is well within the capability of those skilled in the art, especially in light of the disclosure provided herein. The therapeutically effective amount may vary according to a variety of factors such as the subject's condition, weight, sex and age.

In an embodiment, a pharmaceutical composition of the present invention may comprise more than one polypeptide, peptide, conjugate, or a nucleic acid or vector encoding said polypeptide, peptide, or conjugate of the present invention. In an embodiment, the pharmaceutical composition of the present invention may comprise (a) at least one polypeptide, peptide, conjugate, or a nucleic acid or vector encoding said polypeptide, peptide, or conjugate of the present invention; and (b) at least one second pharmaceutical agent or therapeutic agent.

In an embodiment, the second pharmaceutical agent or therapeutic agent may be an anti-inflammatory drug, an immunosuppressant, an antibiotic, an antiviral agent, a small molecule drug, or an antibody. In some embodiments, the second pharmaceutical agent or therapeutic agent is formulated in admixture with, or in a separate pharmaceutical composition from, the at least one polypeptide, peptide, conjugate, or a nucleic acid or vector encoding said polypeptide, peptide, or conjugate, e.g. for simultaneous or for sequential administration.

Pharmaceutical compositions provided herein may be prepared in various pharmaceutical dosage forms, such as an instant release, controlled release, sustained release, or target drug-delivery system. Commonly used dosage forms include, for example, solutions and suspensions, (micro-) emulsions, ointments, gels, creams, pastes, foams, suppositories, ovules, implants, patches, liposomes, tablets, dragees, lozenges, soft or hard shell capsules, amorphous or crystalline powders, effervescent powders or tablets, aerosols, and lyophilized formulations. Depending on the route of administration used, special devices may be required for application or administration of a dosage form, such as syringes and needles, inhalers, pumps, injection pens, applicators, special flasks, or other devices for administration, which may also be implanted within a body. Pharmaceutical dosage forms provided herein may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes.

Pharmaceutical compositions provided herein may further comprise a pharmaceutically acceptable carrier, excipient, and/or stabilizer (Remington: The Science and practice of Pharmacy 20th Ed., 2000, Lippincott Williams and Wilkins, Ed. K. E. Hoover). Acceptable carriers, excipients, or stabilizers are nontoxic to recipients at the dosages and concentrations, and may comprise, for example but not limited to, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical dosage forms provided herein may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes.

### Methods and Uses

Polypeptides, peptides, conjugates, or nucleic acids or vectors encoding said polypeptides, peptides or conjugates, or pharmaceutical compositions of the present invention may be used for inhibiting CCR2 signaling in a cell. In some embodiments, the cell is a dendritic cell, a monocyte, a plasma cell, a macrophage, a Kupffer cell, a Langerhans cell, a T cell, a B cell, an erythroid cell, a hepatic stellate cell, a cholangiocyte, a type 2 alveolar cell, a gastric mucus-secreting cell, an NK cell, a hepatocyte, a Hofbauer cell, a spermatid, a fibroblast, a myeloid-derived suppressor cell, a neutrophil, an osteoclast, a stem cell, a basal keratinocyte, a cardiomyocyte, an endothelial cell, a breast glandular cell, a breast myoepithelial cell, a glandular cell, a luminal cell, a theca cell, a spermatogonium, a cytotrophoblast, a smooth muscle cell, an adipocyte, a CCR2-expressing immortalized cell, or a cancer cell. In some embodiments, the cell is a CCR2-expressing cancer cell from a cancer such as leukemia such as acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia; myelodysplastic syndrome (MDS); a lymphoma such as Hodgkin's disease, a malignant lymphoma, non-Hodgkin's lymphoma, Burkitt's lymphoma; multiple myeloma; Kaposi's sarcoma; colorectal cancer such as colorectal carcinoma; pancreatic cancer such as pancreatic carcinoma; renal cell carcinoma; breast cancer; prostate cancer; cervical cancer; ovarian cancer; liver cancer; kidney cancer; stomach cancer; bladder cancer; ling cancer; oesophageal cancer; nasopharyngeal carcinoma; malignant histiocytosis; paraneoplastic syndrome/hypercalcemia of malignancy; solid tumors; adenocarcinoma such as lung adenocarninoma; squamous cell carcinomas such as squamous epithelial cell and basal squamous epithelial cell; sarcoma such as osteosarcoma; malignant melanoma; melanoma; thyroid carcinoma; salivary adenoid cystic carcinoma, glioma, or haemangioma. In some embodiments, the cancer cell is a cancerous dendritic cell, monocyte, plasma cell, macrophage (e.g. a Kupffer cell or a Langerhans cell), T cell, B cell, NK cell, myeloid-derived suppressor cell, or neutrophil. By “cancerous” cell it is meant that the cell is derived from a normal cell lineage and contains one or more genetic abnormalities leading to transformation into a cancer cell.

Polypeptides, peptides, conjugates, or nucleic acids or vectors encoding said polypeptides, peptides or conjugates, or pharmaceutical compositions of the present invention may be used in the treatment or prevention of diseases or disorders including pulmonary disease, a cancer, an inflammatory or immune-related disease, a cardiovascular disease, a neurologic disease, a fibrotic condition, a wound or tissue injury, or an infectious disease in a subject. In some embodiments, the subject is a human subject.

In an embodiment, the pulmonary disease is pneumonia; lung abscess; occupational lung diseases caused by dust, gas, or mists; asthma; bronchiolitis fibrosa obliterans; respiratory failure; hypersensitivity diseases of the lungs including hypersensitivity pneumonitis (extrinsic allergic alveolitis), allergic bronchopulmonary aspergillosis, and drug reactions; adult respiratory distress syndrome (ARDS); Goodpasture's Syndrome; chronic obstructive airway disorders; chronic obstructive pulmonary disease; idiopathic interstitial lung diseases such as idiopathic pulmonary fibrosis and sarcoidosis; desquamative interstitial pneumonia; acute interstitial pneumonia; respiratory bronchiolitis-associated interstitial lung disease; idiopathic bronchiolitis obliterans with organizing pneumonia; lymphocytic interstitial pneumonitis; Langerhans' cell granulomatosis; idiopathic pulmonary hemosiderosis; acute bronchitis; pulmonary alveolar; proteinosis; bronchiectasis; pleural disorders; atelectasis; cystic fibrosis; or pulmonary embolism.

In an embodiment, the cancer is leukemia such as acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia; myelodysplastic syndrome (MDS); a lymphoma such as Hodgkin's disease, a malignant lymphoma, non-Hodgkin's lymphoma, Burkitt's lymphoma; multiple myeloma; Kaposi's sarcoma; colorectal cancer such as colorectal carcinoma; pancreatic cancer such as pancreatic carcinoma; renal cell carcinoma; breast cancer; prostate cancer; cervical cancer; ovarian cancer; liver cancer; kidney cancer; stomach cancer; bladder cancer; ling cancer; oesophageal cancer; nasopharyngeal carcinoma; malignant histiocytosis; paraneoplastic syndrome/hypercalcemia of malignancy; solid tumors; adenocarcinoma such as lung adenocarninoma; squamous cell carcinomas such as squamous epithelial cell and basal squamous epithelial cell; sarcoma such as osteosarcoma; malignant melanoma; melanoma; thyroid carcinoma; salivary adenoid cystic carcinoma, glioma, or haemangioma. In an embodiment, the polypeptide, peptide, or conjugate as described herein is used for inhibiting CCR2 signalling in a cancer cell, wherein the cancer cell is a cell of a cancer such as, for example, the cancers described above.

In an embodiment, the inflammatory or immune-related disease is rheumatoid arthritis; psoriatic arthritis; ankylosing spondylitis; gastric ulcer; seronegative arthropathies; osteoarthritis; inflammatory bowel disease; ulcerative colitis; systemic lupus erythematosus; antiphospholipid syndrome; iridocyclitis; uveitis; optic neuritis; idiopathic pulmonary fibrosis; systemic vasculitis/Wegener's granulomatosis; sarcoidosis; orchitis; allergic atopic diseases; asthma; allergic rhinitis; allergic conjunctivitis; eczema; dermatitis; allergic conjunctivitis; hypersensitivity pneumonitis; organ transplant rejection; graft-versus-host disease; systemic inflammatory response syndrome; sepsis syndrome; gram positive sepsis; gram negative sepsis; culture negative sepsis; fungal sepsis; neutropenic fever; urosepsis; meningococcaemia; acute pancreatitis; adult respiratory distress syndrome; chronic inflammatory pathologies; sarcoidosis; Crohn's disease; diabetes; nephrosis; diabetic nephropathy; diabetic retinopathy; diabetic retinitis; diabetic microangiopathy; atopic diseases; atopic dermatitis; hypersensitivity reactions; hay fever; perennial rhinitis; conjunctivitis; endometriosis; urticaria; systemic anaphylaxis; haemolytic diseases; Graves disease; Raynaud's disease; myasthenia gravis; antibody-meditated cytotoxicity; type IU hypersensitivity reactions; polyneuropathy; endocrinopathy; monoclonal gammopathy; skin changes syndrome; antiphospholipid syndrome; pemphigus; scleroderma; mixed connective tissue disease; idiopathic Addison's disease; chronic active hepatitis; non-alcoholic steatohepatitis; liver fibrosis; liver cirrhosis; non-alcoholic fatty liver disease; primary biliary cirrhosis; primary sclerosing cholangitis; vitiligo; vasculitis; gingivitis; periodontitis; gum disease; post-MI cardiotomy syndrome; type IV hypersensitivity; hypersensitivity pneumonitis; granulomas due to intracellular organisms; drug sensitivity; Wilson's disease; hemochromatosis; thyroiditis such as Hashimoto's thyroiditis; primary biliary cirrhosis; encephalomyelitis; cachexia; cystic fibrosis; neonatal chronic lung disease; chronic obstructive pulmonary disease (COPD); familial hemophagocytic lymphohistiocytosis; dermatologic conditions such as psoriasis and alopecia; nephrotic syndrome; nephritis such as glomerular nephritis; acute renal failure; opthalmatic disorders; preeclampsia; or effects from a therapy including OKT3 therapy, anti-CD3 therapy, cytokine therapy, chemotherapy, radiation therapy.

In an embodiment, the cardiovascular disease is cardiac stun syndrome; myocardial infarction; congestive heart failure; stroke; ischemic stroke; haemorrhage; atherosclerosis; restenosis; angiostenosis; diabetic atherosclerotic disease; hypertension; arterial hypertension; renovascular hypertension; syncope; shock; syphilis of the cardiovascular system; heart failure; cor pulmonale; primary pulmonary hypertension; cardiac arrhythmias; atrial ectopic beats; atrial flutter; atrial fibrillation (sustained or paroxysmal); post perfusion syndrome; cardiopulmonary bypass inflammation response; chaotic or multifocal atrial tachycardia; regular narrow QRS tachycardia; ventricular fibrillation; His bundle arrythmias; atrioventricular block; bundle branch block; myocardial ischemic disorders; myocarditis; coronary artery disease; angina pectoris; cardiomyopathy such as dilated congestive cardiomyopathy, restrictive cardiomyopathy, diastolic cardiomyopathy; valvular heart diseases; endocarditis; pericardial disease; aortic and peripheral aneurysms; inflammation of the aorta; occlusion of the abdominal aorta and its branches; peripheral vascular disorders; occlusive arterial disorders; peripheral atherosclerotic disease; thromboangitis obliterans; functional peripheral arterial disorders; acrocyanosis; erythromelalgia; venous diseases; venous thrombosis; varicose veins; arteriovenous fistula; lymphedema; lipoedema; unstable angina; reperfusion injury; post pump syndrome; ischemia-reperfusion injury; or reperfusion disorders.

In an embodiment, the neurologic disease is inflammatory pain; chronic pain; neuropathic pain such as low back pain, hip pain, leg pain; neuralgia such as post-traumatic neuralgia and post herpetic neuralgia; diabetic neuropathy; nerve injury-induced pain; acquired immune deficiency syndrome (AIDS) related neuropathic pain; toxin and chemotherapy caused nerve injuries; phantom limb pain; root avulsions; painful traumatic mononeuropathy; painful polyneuropathy; thalamic pain syndrome; post-stroke pain; central nervous system injury; post surgical pain; carpal tunnel syndrome; trigeminal neuralgia; post mastectomy syndrome; post-thoracotomy syndrome; stump pain; repetitive motion pain; neuropathic pain associated hyperalgesia and allodynia; neurodegenerative diseases; migraine headache; demyelinating diseases such as multiple sclerosis and acute transverse myelitis; extrapyramidal and cerebellar disorders such as lesions of the corticospinal system; disorders of the basal ganglia or cerebellar disorders; hyperkinetic movement disorders such as Huntington's chorea and senile chorea; drug-induced movement disorders such as those induced by drugs which block CNS dopamine receptors; hypokinetic movement disorders such as Parkinson's disease; Progressive supranuclear palsy; structural lesions of the cerebellum; spinocerebellar degenerations such as spinal ataxia, Friedreich's ataxia, cerebellar cortical degenerations, multiple system degenerations (Mencel, Dejerine-Thomas, Shy-Drager, and Machado-Joseph); disorders of the motor unit such as neurogenic muscular atrophies (anterior horn cell degeneration, such as amyotrophic lateral sclerosis, infantile spinal muscular atrophy and juvenile spinal muscular atrophy); Alzheimer's disease; diffuse Lewy body disease; senile dementia of Lewy body type; Wernicke-Korsakoff syndrome; Creutzfeldt-Jakob disease; subacute sclerosing panencephalitis; Hallerrorden-Spatz disease; or dementia pugilistica.

In an embodiment, the fibrotic condition is liver fibrosis; viral-induced cirrhosis; autoimmune-induced hepatitis; lung fibrosis such as idiopathic pulmonary fibrosis; kidney fibrosis such as scleroderma, diabetic nephritis, glomerular nephritis, lupus nephritis; dermal fibrosis such as scleroderma, hypertrophic and keloid scarring, burns; myelofibrosis; Neurofibromatosis; fibroma; intestinal fibrosis; and fibrotic adhesions resulting from surgical procedures.

In an embodiment, the wound or tissue injury is bodily injury or a trauma associated with surgery including thoracic, abdominal, cranial, or oral surgery; acute traumatic wounds; ischemic ulcers; pressure sores; fistulae; thermal burns; donor site wounds; or aphthous wounds.

In an embodiment, the infectious disease is bacterial infection; parasitic infection; fungal infection; HIV infection; meningitis; hepatitis A, B or C; septic arthritis; peritonitis; pneumonia; epiglottitis; malaria; dengue haemorrhagic fever; leishmaniasis; leprosy; toxic shock syndrome; streptococcal myositis; mycobacterium infection; Pneumocystis carinii pneumonia, pelvic inflammatory disease; orchitis; epididymitis; legionella; Lyme disease; influenza; Epstein Barr virus; vital-associated hemophagocytic syndrome; or encephalitis.

The present invention provides the use of polypeptides, peptides, conjugates, or nucleic acids or vectors encoding said polypeptides, peptides or conjugates, or pharmaceutical compositions of the present invention for the treatment and/or prophylaxis (prevention) of diseases or disorders that may be treated by modulating the activity of CCR2 or modulating the activity of cells expressing CCR2. Moreover, conjugates of the present invention may also be used to carry moieties into cells expressing CCR2. In some embodiments, the moiety is an immunoglobulin domain, a chemokine domain, a toxin, a carrier protein, a polymer, a lipid, or a detectable marker.

One or more of the polypeptides, peptides, conjugates, or nucleic acids or vectors encoding said polypeptides, peptides or conjugates of the present invention may be administered to a subject. When more than one is administered, they may be administered together (as an admixture or separately though substantially simultaneously) or sequentially. They may be administered in combination with one or more other pharmaceutical or therapeutic agent that are not comprised within the polypeptides, peptides, conjugates, or nucleic acids or vectors encoding said polypeptides, peptides or conjugates of the present invention. The polypeptides, peptides, conjugates, or nucleic acids or vectors encoding said polypeptides, peptides or conjugates of the present invention may then also be administered together (as an admixture or separately though substantially simultaneously) with said one or more other pharmaceutical or therapeutic agent, or sequentially.

“Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression, suppression of disease, delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject.

“Treating” may be distinguished from “preventing” in that “treating” typically occurs in a subject who already has a disease or disorder, or is known to have already been exposed to an infectious agent, whereas “preventing” typically occurs in a subject who does not have a disease or disorder, or is not known to have been exposed to an infectious agent. As will be appreciated, there may be overlap in treatment and prevention. For example, it is possible to be “treating” a disease in a subject, while at same time “preventing” symptoms or progression of the disease.

### Modes of Administration

The polypeptides, peptides, conjugates, or nucleic acids or vectors encoding said polypeptides, peptides or conjugates of the present invention can be delivered directly or in pharmaceutical compositions containing carriers, excipients, and/or stabilizers, as is known in the art. The present methods of treatment include administration of a therapeutically effective amount of the polypeptides, peptides, conjugates, or nucleic acids or vectors encoding said polypeptides, peptides or conjugates of the present invention to a subject.

For the methods and uses of the present invention, polypeptides, peptides, conjugates, or nucleic acids or vectors encoding said polypeptides, peptides, conjugates, or pharmaceutical compositions of the present invention may be administered to a subject by conventional techniques, such as intravenously (as a bolus or by continuous infusion over a period of time), intramuscularly, transmucosally, intraperitoneally, intra-cerebrally, subcutaneously, intra-articularly, intrasynovially, intrathecally, nasally, orally, topically, or by inhalation. Other suitable administration routes may include intra-lesional or peri-lesional routes.

For intravenous injection, polypeptides, peptides, conjugates, or nucleic acids or vectors encoding said polypeptides, peptides, conjugates, or pharmaceutical compositions of the present invention may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, pharmaceutical compositions provided herein may be formulated in liquid or solid dosage forms and optionally as instant or controlled/sustained release formulations. Suitable dosage forms for oral ingestion by a subject include tablets, capsules, pills, dragees, hard and soft shell capsules, liquids, gels, syrups, slurries, suspensions, and emulsions.

Solid oral dosage forms can be obtained using excipients, which may include inert diluents, fillers, disintegrants, binders (dry and wet), dissolution retardants, lubricants, glidants, antiadherants, cationic exchange resins, wetting agents, antioxidants, preservatives, colouring, sweetening and flavouring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicon dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule

Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which an active ingredient is mixed with a solid diluent and soft gelatine capsules wherein the active ingredient is mixed with water or an oil such as peanut oil, liquid paraffin or olive oil.

In some embodiments, polypeptides, peptides, conjugates, or nucleic acids or vectors encoding said polypeptides, peptides, conjugates, or pharmaceutical compositions of the present invention may be administered topically, via the skin or mucous membrane, such as through a skin patch, a semi-solid or a liquid formulation, for example a gel, a (micro-) emulsion, an ointment, a solution, a (nano/micro)-suspension, or a foam. The penetration of an active ingredient into the skin or mucous membrane and underlying tissues of a subject can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents.

In some embodiments, polypeptides, peptides, conjugates, or nucleic acids or vectors encoding said polypeptides, peptides, conjugates, or pharmaceutical compositions of the present invention may be administered by inhalation, or to the nose, in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of an active ingredient and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. Pharmaceutical compositions suitable for parenteral administration may take such forms as suspensions, solutions, or emulsions in oily or aqueous vehicles, and may contain buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Formulations, providing controlled or sustained release of an active agent, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other sustained delivery systems may be presented in form of implants and pumps requiring incision.

Suitable carriers for intravenous injection are well-known in the art and include water-based solutions containing a base, such as, for example, sodium hydroxide, to form an ionized agent, sucrose or sodium chloride as a tonicity agent. A water-based solution may comprise a buffer containing phosphate or histidine. Co-solvents, such as polyethylene glycols, may be added. These water-based systems are effective at dissolving agents and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

Particular embodiments of the disclosure include, without limitation, the following:
1. A polypeptide comprising an N-terminal portion and a C-terminal portion, wherein the N-terminal portion comprises an amino acid sequence with 0, 1, 2, 3, or 4 amino acid substitutions relative to any one of SEQ ID NO: 40-70 and 76-145, and wherein the C-terminal portion comprises an amino acid sequence at least 70% identical to SEQ ID NO: 71, 72, 74, or 75.
2. The polypeptide of embodiment 1, wherein the amino acid substitution is a conservative substitution.
3. The polypeptide of embodiment 1, wherein the N-terminal portion comprises the amino acid sequence of any one of SEQ ID NO: 40-70 and 76-145.
4. The polypeptide of embodiment 1, wherein the N-terminal portion comprises the amino acid sequence FTNPTWXXXX (SEQ ID NO: 146), wherein X is any amino acid.
5. The polypeptide of embodiment 4, wherein the N-terminal portion comprises the amino acid sequence FTNPTW[A or D or R or S or K or Q][P or A or T or G or S or Q or R or H or E][V or F or Q or G or S or H or L or Y][T or V or Q or S or A] (SEQ ID NO: 147).
6. The polypeptide of embodiment 4, wherein the N-terminal portion comprises the amino acid sequence of any one of SEQ ID NO: 40 and 76-99.
7. The polypeptide of embodiment 1, wherein the N-terminal portion comprises the amino acid sequence FPX₁DGWX₂X₃X₄X₅(SEQ ID NO: 148), wherein X₁ is Methionine or Norleucine, and X₂-X₅ is any amino acid.
8. The polypeptide of embodiment 7, wherein the N-terminal portion comprises the amino acid sequence FPX₁DGW[A or R or G or H or V or Q][P or S or G or E][V or R or L or E or T or G or Q][T or V or Q] (SEQ ID NO: 149), wherein X₁ is Methionine or Norleucine.
9. The polypeptide of embodiment 7, wherein the N-terminal portion comprises the amino acid sequence of any one of SEQ ID NO: 41 and 100-145.
10. The polypeptide of any one of embodiments 1-4, wherein the N-terminal portion consists of 8-11 amino acids.
11. The polypeptide of any one of embodiments 1-5, wherein the C-terminal portion comprises the amino acid sequence of SEQ ID NO: 71, 72, 74, or 75.
12. The polypeptide of any one of embodiments 1-11, wherein the polypeptide inhibits CCR2 with an IC50 of less than 300 nM, less than 200 nM, less than 100 nM, less than 50 nM, less than 30 nM, less than 10 nM, or less than 3 nM.
13. A peptide comprising an amino acid sequence with 0, 1, 2, 3, or 4 amino acid substitutions relative to any one of SEQ ID NO: 40-70 and 76-145.
14. The peptide of embodiment 13, wherein the amino acid substitution is a conservative substitution.
15. The peptide of embodiment 13, comprising the amino acid sequence of any one of SEQ ID NO: 40-70 and 76-145.
16. The peptide of embodiment 13, comprising the amino acid sequence FTNPTWXXXX (SEQ ID NO: 146), wherein X is any amino acid.
17. The peptide of embodiment 16, comprising the amino acid sequence FTNPTW[A or D or R or S or K or Q][P or A or T or G or S or Q or R or H or E][V or F or Q or G or S or H or L or Y][T or V or Q or S or A] (SEQ ID NO: 147).
18. The peptide of embodiment 16, comprising the amino acid sequence of any one of SEQ ID NO: 40 and 76-99.
19. The peptide of embodiment 13, comprising the amino acid sequence FPX₁DGWX₂X₃X₄X₅ (SEQ ID NO: 148), wherein X₁ is Methionine or Norleucine, and X₂-X₅ is any amino acid.
20. The peptide of embodiment 13, comprising the amino acid sequence FPX₁DGW[A or R or G or H or V or Q][P or S or G or E][V or R or L or E or T or G or Q][T or V or Q] (SEQ ID NO: 149), wherein X₁ is Methionine or Norleucine.
21. The peptide of embodiment 13, comprising the amino acid sequence of any one of SEQ ID NO: 41 and 100-145.
22. A nucleic acid molecule encoding the polypeptide of any one of embodiments 1-12 or the peptide of any one of embodiments 13-21.
23. A vector comprising the nucleic acid molecule of embodiment 22.
24. A host cell comprising the nucleic acid molecule of embodiment 22 or the vector of embodiment 23.
25. A pharmaceutical composition comprising the polypeptide of any one of embodiments 1-12, the peptide of any one of embodiments 13-21, the nucleic acid molecule of embodiment 22, or the vector of embodiment 23, and a pharmaceutically acceptable carrier, excipient, and/or stabilizer.
26. The pharmaceutical composition of embodiment 25, further comprising a therapeutic agent.
27. The polypeptide of any one of embodiments 1-12, the peptide of any one of embodiments 13-21, the nucleic acid molecule of embodiment 22, the vector of embodiment 23, or the pharmaceutical composition of embodiment 25 or 26 for use in inhibiting CCR2 signaling in a cell.
28. The polypeptide, peptide, nucleic acid molecule, vector, or pharmaceutical composition of embodiment 27, wherein the cell is a dendritic cell, a monocyte, a plasma cell, a macrophage, a Kupffer cell, a Langerhans cell, a T cell, a B cell, an erythroid cell, a hepatic stellate cell, a cholangiocyte, a type 2 alveolar cell, a gastric mucus-secreting cell, an NK cell, a hepatocyte, a Hofbauer cell, a spermatid, a fibroblast, a myeloid-derived suppressor cell, a neutrophil, an osteoclast, a stem cell, a basal keratinocyte, a cardiomyocyte, an endothelial cell, a breast glandular cell, a breast myoepithelial cell, a glandular cell, a luminal cell, a theca cell, a spermatogonium, a cytotrophoblast, a smooth muscle cell, an adipocyte, a CCR2-expressing immortalized cell, or a cancer cell.
29. The polypeptide, peptide, nucleic acid molecule, vector, or pharmaceutical composition of embodiment 27 or 28, wherein the cell is in in vitro.
30. The polypeptide, peptide, nucleic acid molecule, vector, or pharmaceutical composition of embodiment 27 or 28, wherein the cell is in a subject.
31. The polypeptide of any one of embodiments 1-12, the peptide of any one of embodiments 12-21, the nucleic acid molecule of embodiment 22, the vector of embodiment 23, or the pharmaceutical composition of embodiment 25 or 26 for use in treating or preventing a disease or disorder associated with CCR2 signaling in a subject.
32. The polypeptide, peptide, nucleic acid molecule, vector, or pharmaceutical composition of embodiment 31, wherein the disease or disorder is a pulmonary disease, a cancer, an inflammatory or immune-related disease, a cardiovascular disease, a neurologic disease, a fibrotic condition, a wound or tissue injury, or an infectious disease.
33. A method of inhibiting CCR2 signaling in a cell, comprising contacting the cell with the polypeptide of any one of embodiments 1-12, the peptide of any one of embodiments 13-21, the nucleic acid molecule of embodiment 22, the vector of embodiment 23, or the pharmaceutical composition of embodiment 25 or 26.
34. The method of embodiment 33, wherein the cell is a dendritic cell, a monocyte, a plasma cell, a macrophage, a Kupffer cell, a Langerhans cell, a T cell, a B cell, an erythroid cell, a hepatic stellate cell, a cholangiocyte, a type 2 alveolar cell, a gastric mucus-secreting cell, an NK cell, a hepatocyte, a Hofbauer cell, a spermatid, a fibroblast, a myeloid-derived suppressor cell, a neutrophil, an osteoclast, a stem cell, a basal keratinocyte, a cardiomyocyte, an endothelial cell, a breast glandular cell, a breast myoepithelial cell, a glandular cell, a luminal cell, a theca cell, a spermatogonium, a cytotrophoblast, a smooth muscle cell, an adipocyte, a CCR2-expressing immortalized cell, or a cancer cell.
35. The method of embodiment 34, wherein the cell is in vitro.
36. The method of embodiment 34, wherein the cell is in a subject.
37. A method of treating or preventing a disease or disorder associated with CCR2 signaling in a subject, comprising administering to the subject the polypeptide of any one of embodiments 1-12, the peptide of any one of embodiments 13-21, the nucleic acid molecule of embodiment 22, the vector of embodiment 23, or the pharmaceutical composition of embodiment 25 or 26.
38. The method of embodiment 37, wherein the disease or disorder is a pulmonary disease, a cancer, an inflammatory or immune-related disease, a cardiovascular disease, a neurologic disease, a fibrotic condition, a wound or tissue injury, or an infectious disease.
39. Use of the polypeptide of any one of embodiments 1-12, the peptide of any one of embodiments 13-21, the nucleic acid molecule of embodiment 22, the vector of embodiment 23, or the pharmaceutical composition of embodiment 25 or 26 for inhibiting CCR2 signaling in a cell.
40. The use of embodiment 39, wherein the cell is a dendritic cell, a monocyte, a plasma cell, a macrophage, a Kupffer cell, a Langerhans cell, a T cell, a B cell, an erythroid cell, a hepatic stellate cell, a cholangiocyte, a type 2 alveolar cell, a gastric mucus-secreting cell, an NK cell, a hepatocyte, a Hofbauer cell, a spermatid, a fibroblast, a myeloid-derived suppressor cell, a neutrophil, an osteoclast, a stem cell, a basal keratinocyte, a cardiomyocyte, an endothelial cell, a breast glandular cell, a breast myoepithelial cell, a glandular cell, a luminal cell, a theca cell, a spermatogonium, a cytotrophoblast, a smooth muscle cell, an adipocyte, a CCR2-expressing immortalized cell, or a cancer cell.
41. The use of embodiment 40, wherein the cell is in vitro.
42. The use of embodiment 40, wherein the cell is in a subject.
43. Use of the polypeptide of any one of embodiments 1-12, the peptide of any one of embodiment 13-21, the nucleic acid molecule of embodiment 22, the vector of embodiment 23, or the pharmaceutical composition of embodiment 25 or 26 for treating or preventing a disease or disorder associated with CCR2 signaling in a subject.
44. The use of embodiment 43, wherein the disease or disorder is a pulmonary disease, a cancer, an inflammatory or immune-related disease, a cardiovascular disease, a neurologic disease, a fibrotic condition, a wound or tissue injury, or an infectious disease.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

All publications and patents cited herein are incorporated by reference in their entirety as if each individual publication or patent were specifically and individually indicated to be incorporated by reference. In the case of any conflict between a definition of a term in the present disclosure and a definition in a cited publication or patent, the definition provided in the present disclosure is to be used in describing the present invention.

The present invention will now be described by way of non-limiting examples having regard to the appended drawings.

### EXAMPLES

### Materials and Methods

CCR2 inhibitory potency assay was determined using the calcium flux method.

Ca²⁺ flux measurements were performed using an FDSS micro-cell device (HAMAMATSU). On the day of the experiment, THP-1 cells were seeded 20000 cells/well) to wells of black-walled clear-bottom 384-well plates.

Synthesized test samples (100 mM) were diluted in PBS supplemented with 1% BSA and 25 mM HEPES to generate dilution series for dose-response experiments: 12-point dose-response starting at 688 nM with a 2.5-fold dilution interval for each treatment).

THP-1 cells were loaded with a calcium-sensitive fluorescent dye (Screen Quest™ Fluo-8 No Wash Calcium Assay Kit, AAT Bioquest) according to the manufacturer's instructions, then a first addition of either test sample dilutions or vehicle alone were added. 5 minutes later, cells were stimulated with 100 nM CCL2.

Fluorescence signals (ex. 490 nm, em. 525 nm) were recorded during the full course of the experiment.

### Example 1: Synthesis of Fmoc-Thr(tBu)-2-chlorotrityl Resin

Synthesis of polypeptides and peptides according to the present invention, such as the exemplary polypeptide 1P2-CCL2 (Met64Nle OB-004), involved synthesis of Fmoc-Thr(tBu)-2-chlorotrityl resin.

The synthesis of 1P2-CCL2 (Met64Nle OB-004) is detailed by example. 3 g of 2-chlorotrityl resin (2CT resin loading: 1.08 mmol/g) was weighed in a 50 mL peptide synthesis fritted syringe and swelled with 20 mL of DMF for 30 min at room temperature (RT). Fmoc-Thr(tBu)-OH (0.15 mmol, 178.9 mg) and DIPEA (392 μL, 0.45 mmol, 5 eq to amino acid) were dissolved in 12 mL of DMF and added to the pre-swelled resin, and the reaction mix was rotated at 6 rpm for 1.5 h at RT. The loading of the resin was determined by quantifying the Fmoc-Thr(tBu)-OH in the flow through using UV absorbance (UV 301 nm; ε 7800 M−1 cm−1), and was quantified at 0.149 mmol/g. After that, 1 mL of DIPEA (2 eq to the resin) and 1.5 mL MeOH were added to the reaction mixture sequentially and the reaction was rotated at 6 rpm for 10 min at RT to quench the unreacted trityl chloride-groups of the resin. Then the resin was washed three times with 12 mL of DMF and two times with 12 mL of MeOH and lyophilized.

### Example 2: Synthesis and Purification of 1P2-CCL2(Met64Nle OB-004) Core Fragment (11-76)

A C-terminal fragment of human CCL2 [11-76 human CCL2], in which the methionine residue at position 64 relative to SEQ ID NO: 1) was replaced by norleucine (Nle), was synthesized on an automated peptide synthesizer (Prelude®, Protein Technologies, Inc.) at a 100 μmol scale on Fmoc-Thr(tBu)-2CT-resin (0.667 g; loading 0.15 mmol/g) using Fmoc-chemistry. Coupling was carried out twice for each amino acid [4 equivalents (equiv)] using HCTU (4 equiv) and DIPEA (10 equiv) in 4.5 mL of N,N′-dimethylformamide (DMF) with nitrogen purging for 45 min. The resin was washed once with 4 mL of DMF, and unreacted amino groups were capped by incubation with 5% acetic anhydride and 0.5 M DIPEA in 8 mL of DMF with nitrogen purging for 5 min. The resin was washed nine times with 4 mL of DMF. Fmoc groups were removed by incubation twice with 4 mL of 20% (v/v) piperidine in DMF with nitrogen purging for 10 min. The resin was washed nine times with 4 mL of DMF. In all washing steps, the resin was purged with nitrogen for 30 seconds. At the end of the synthesis, the resin was washed three times with 8 mL of methanol manually and air dried for 1 h before peptide cleavage.

Peptides were cleaved from the resin, and the protecting groups were removed under reducing conditions by incubation in 30 mL of cleavage solution [86% trifluoroacetic acid (TFA), 5% H2O, 5% phenol, and 4% triisopropylsilane (TIS)] with shaking for 4 hours at room temperature (RT). The resin was removed by filtration and filtrate was distributed into 6 falcons (5 mL each). Peptide was precipitated with cold diethyl ether (45 mL), incubated for overnight at −20° C., and pelleted by centrifugation at 4000 g for 20 min.

The core fragment peptide was purified with a reverse phase high-performance liquid chromatography (HPLC) system (Prep 1525 HPLC, Waters) using a preparative C8 reversed-phase column (10-15 μm, 250×22 mm; Vydac 208TP, GRACE), applying a flow rate of 15 mL/min and a linear gradient of 25 to 40% (v/v) solvent B for 30 min [solvent A: H2O with 0.1% (v/v) TFA; solvent B: 90% (v/v) acetonitrile (ACN)/H2O with 0.1% (v/v) TFA]. Fractions containing the desired peptide were lyophilized.

### Example 3: Synthesis of 1P2-CCL2(Met64Nle OB-004) N-Terminal Fragment (1-10)

The N-terminal fragment of 1P2-CCL2 (Met64Nle OB-004) was synthesized on an Intavis MultiPep RSi parallel peptide synthesizer by standard Fmoc chemistry on Fmoc-Cys(Trt)-SEA-2CP resin [loading 0.136 mmol/g; 14.7 mg/well; scale 2 μmol; SEA=bis(2-SulfanylEthyl)Amino]. Coupling was carried out twice for each amino acid using HCTU and DIPEA (10 equiv) in 59 μL of N,N′-dimethylformamide (DMF) without shaking for 30 min. The unreacted amino groups were capped by incubation with 5% acetic anhydride and 6% 2,6-lutidine in 50 μL of DMF for 10 min. The resin was washed six times with 150 μL of DMF, and Fmoc groups were removed by incubation twice with 50 μL of 20% (v/v) piperidine in DMF for 10 min. The resin was washed ten times with 150 μL of DMF. At the end of the synthesis, the resin was washed ten times with 150 μL of MeOH.

The peptide was cleaved from the resin, and the protecting groups were removed under reducing conditions by incubation in 0.4 mL of cleavage solution (86% TFA, 5% H2O, 5% phenol, and 4% TIS) with shaking at 400 rpm for 3 hours at RT. The peptide was then precipitated with cold diethyl ether (1 mL), incubated for overnight at −20° C., and pelleted by centrifugation at 1700 g for 30 min. This process was repeated three times to remove cleavage impurities, then the pellet was air-dried for 1 hour at RT. The pellet was then dissolved in 1 mL of 70% ACN/H2O (0.1% TFA) solution with the resin removed by filtration. The collected peptide solution was lyophilized, and the mass of the product was confirmed by MALDI-TOF/TOF using DHB (2,5-Dihydroxybenzoic acid) matrix with linear positive ionization method.

### Example 4: Fragment Assembly and Purification of Final Product

4.0 mg of C-terminal core fragment [0.47 μmol] was added to 18 equiv (8 μmol) of N-terminal SEA-peptide fragment in 470 μL ligation buffer (Final concentration of the core fragment=˜1 mM) and incubated the reaction at 37° C. with stirring for overnight [Ligation buffer: 0.2 M sodium phosphate buffer which contains 6 M guanidine hydrochloride (Gn·HCl), 0.1 M TCEP·HCl, 0.1 M MPAA and 50 mM methionine, pH 7.5]. Completion of the reaction was monitored by RP-HPLC. After completion of the reaction, the reaction mixture treated with 240 μL of 0.28 M TCEP·HCl solution (6 M Gn·HCl, pH 5.5) for 30 min at RT and then crude product was purified on preparative HPLC using C8 column [Gradient method: 10-50% solvent B in 80 min & Flow rate: 15 mL/min]. Pure fractions were collected and lyophilized. Ligation yield was 1.4 mg.

For folding, purified linear peptide was dissolved in folding buffer at a concentration of 0.2 mg/mL and left the solution at RT for 2-3 days with stirring (Folding buffer: 2 M Gn·HCl, 0.1 M Tris, 10 mM methionine, 0.5 mM GSH, 0.3 mM oxidized GSH, pH 8.0). The progress of the reaction monitored by RP-HPLC. After completion of the reaction, the reaction mixture acidified with 1 mL of 33% acetic acid solution and then crude product was purified on HPLC using semipreparative C8 column [Gradient method: 10-50% solvent B in 40 min & Flow rate: 4 mL/min]. Pure fractions were collected and lyophilized.

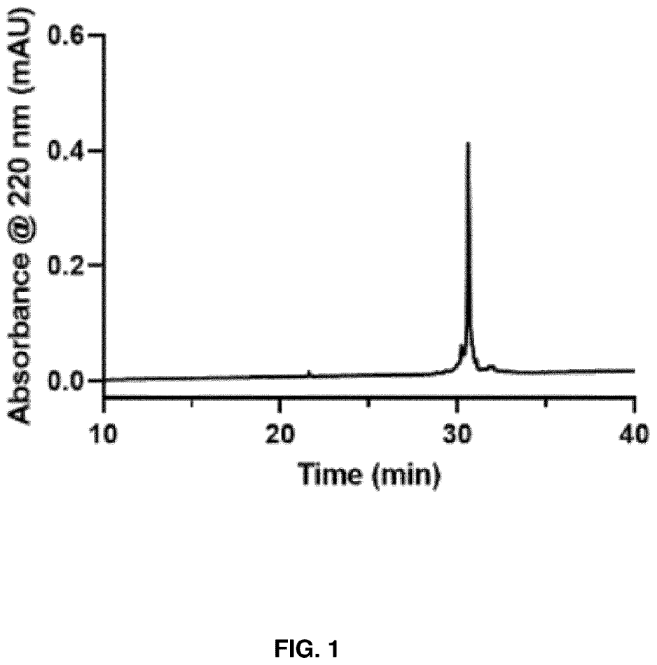

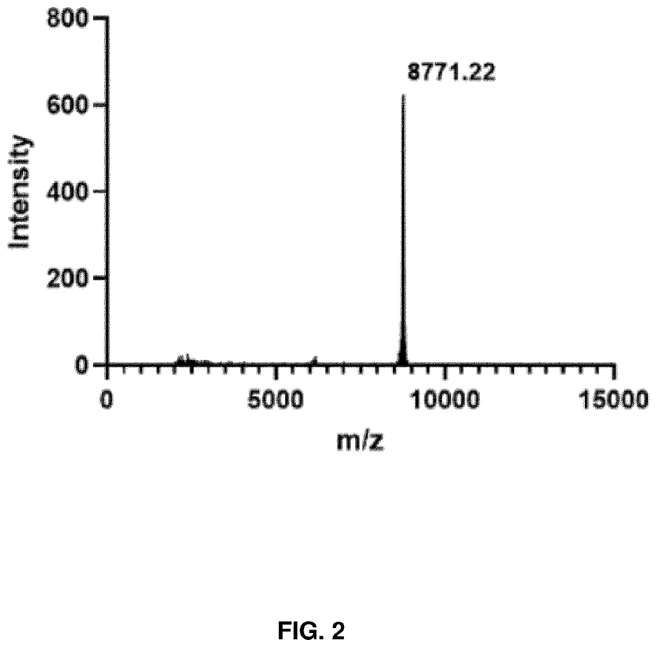

Following final authentication by analytical RP-HPLC (FIG. 1) and mass spectrometry (FIG. 2 wherein MALDI-TOF/TOF m/z calc. avg. mass [M+H]+ 8772.15 and obs. avg. mass [M+H]+ 8771.22), lyophilized 1P2-CCL2 (Met64Nle OB-004) was dissolved in H2O to give a final concentration of 100 μM.

### Example 5: CCR2 Inhibitory Potency Assay

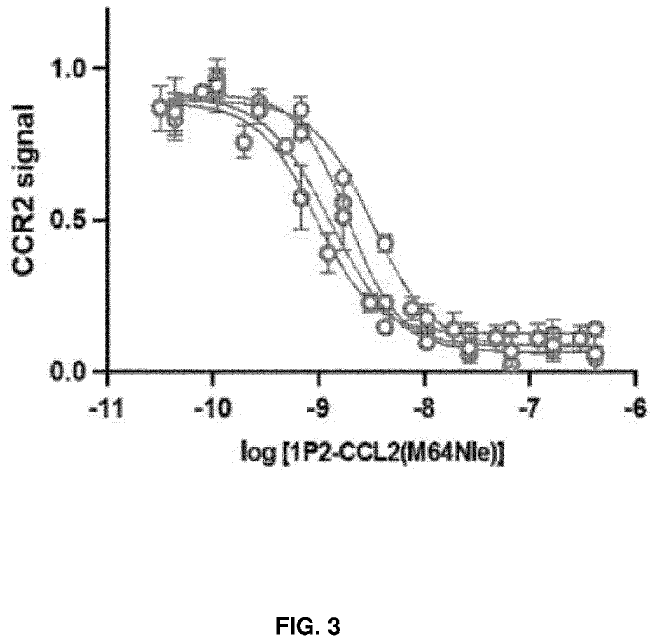

The synthetic sample of 1P2-CCL2(Met64Nle OB-004) was tested in the CCR2 inhibitory potency assay using the calcium flux method described above. FIG. 3 illustrates dose-inhibition curves obtained for 1P2-CCL2(Met64Nle OB-004) in four independent experiments. Data points indicate mean fluorescence signal±s.e.m (n=3). Across the four experiments, fitted IC₅₀ values ranged from 0.9 to 3.2 nM.

### Example 6: Comparative CCR2 Inhibitory Potency Assay

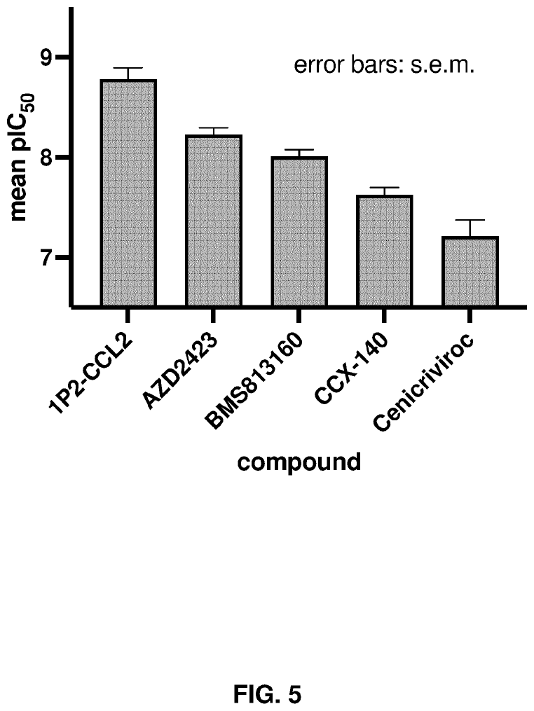

FIGS. 4 and 5 show the results of CCR2 inhibitory potency assays using the calcium flux method described above, testing four known small molecule CCR2 inhibitors against the exemplary CCL2 variant polypeptide 1P2-CCL2 (OB-004). FIG. 4 shows dose-inhibition curves from a representative experiments and FIG. 5 shows mean pIC₅₀ values determined from dose-inhibition curves obtained in four independent experiments.

FIG. 4 illustrates the inhibitory profiles of four known small molecule CCR2 inhibitors and the exemplary CCL2 variant polypeptide 1P2-CCL2 (Met64 Nle OB-004). The four known small molecule CCR2 inhibitors were: AZD2423; BMS-813160; CCX-140; and Cenicriviroc.

FIG. 5 indicates the compiled data from four experiments showing the mean pIC₅₀ for each of the five inhibitors. The compiled data shows that the most potent inhibitor was 1P2-CCL2 (1.7 nM). AZD2423 (6.0 nM) was 3.53-fold less potent than 1P2-CCL2. BMS-813160 (9.8 nM) was 5.76-fold less potent than 1P2-CCL2. CCX-140 (24.0 nM) was 14.12-fold less potent than 1P2-CCL2. Cenicriviroc (62.0 nM) was 36.47-fold less potent than 1P2-CCL2.

The results of four experiments consistently and significantly show that an exemplary CCL2 variant polypeptide of the present invention, 1P2-CCR2 (Met64Nle OB-004), comprising the N-terminal peptide FTNPTWAPVT (SEQ ID NO: 40) is more potent than the known small molecule CCR2 inhibitors tested.

### Example 7: Capture and Transmigration Assay

A capture/transmigration assay using human uterine vascular endothelial cells (HUVECS) was used to provide data relating to monocyte capture, adhesion and transmigration using a HUVEC model.

In the assay, HUVECS are introduced into a chamber slide where they are grown to confluence. The HUVECS are then stimulated with TNF-alpha for either 4 or 18 hours. The HUVEC monolayer is washed with cell culture fluid and preincubated with the experimental compound for 20 mins. Donor blood is collected and monocytes are purified. The purified monocyte suspension is pumped across the HUVEC monolayer using a calibrated pump. Monocyte capture and adhesion are recorded (6 mins) using a camera and microscope operatively connected to image analytic software (not shown). Some monocytes roll across the HUVEC monolayer prior to adhering, while other monocytes do not adhere and are removed from the chamber slide. Cell culture wash media is circulated through the chamber slide. Monocyte capture and transmigration are recorded at 10, 15, 20, 25, 30, 35, 40 and 60 min timepoints. Some monocytes adhere to the HUVEC monolayer, others partially transmigrate through the monolayer, and other monocytes completely transmigrate through the monolayer. Capture and/or transmigration of the monocytes is mediated by CCL2-CCR2 interactions between the monocytes and the HUVECS. Decreases in capture and/or transmigration indicate increased CCR2 inhibition.

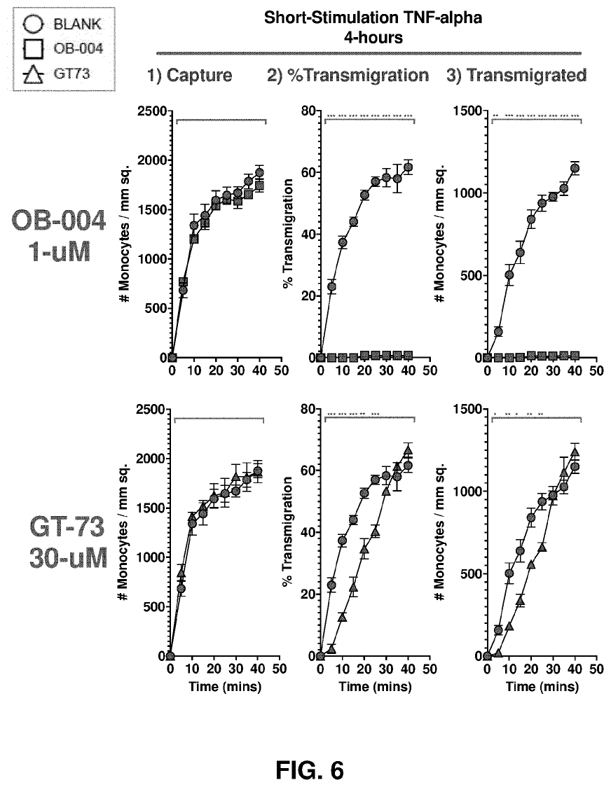

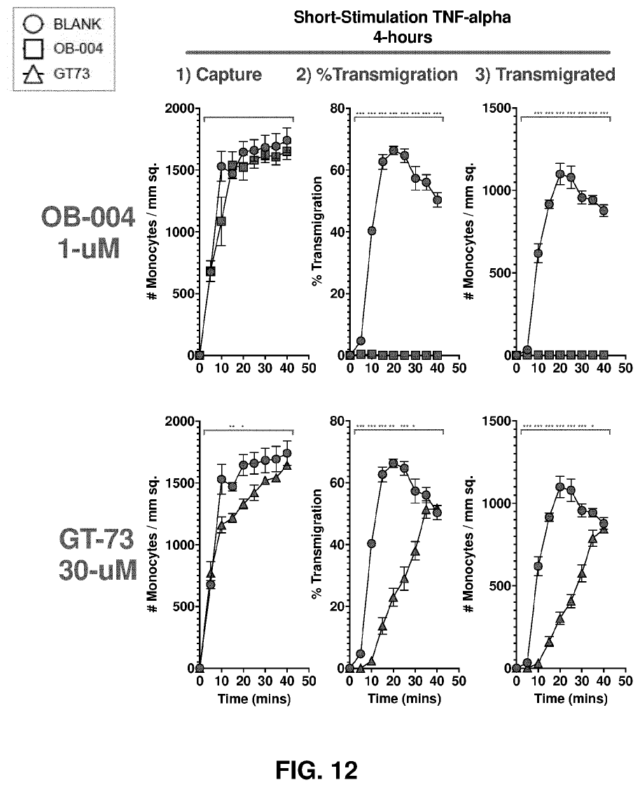

The test parameters for results obtained and illustrated in FIGS. 6-18 were obtained using: a Blank as a negative control; 1P2-CCL2 (Met64Nle OB-004) (1 uM) as the experimental compound; and GT-73 (30 uM) as a positive control. GT-73 blocks leukocyte transendothelial migration by interfering with the function of the cell adhesion molecule PECAM-1 (CD31).

The stages of monocyte adhesion and transmigration are as follows. Suspended monocytes settle onto the HUVEC monolayer. Some monocytes roll prior to adhering, and then locate a cell-cell contact point. Some monocytes then begin to transmigrate between a cell-cell contact point in the monolayer, and eventually become completely transmigrated to the other side of the HUVAC monolayer. Two HUVEC stimulations models were tested: a short stimulation period of 4 hrs with TNF-alpha; and a long stimulation period of 18 hrs with TNF-alpha. Three steps in monocyte trafficking were measured: capture, indicated as (1); % transmigration indicated as (2); and transmigration indicated as (3).

FIG. 6 illustrates the number of monocytes, from Donor 1, captured; % transmigrated and transmigrated on acute activated HUVECS after 4 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 Met64Nle (OB-004) caused a blockade of % transmigration and transmigration.

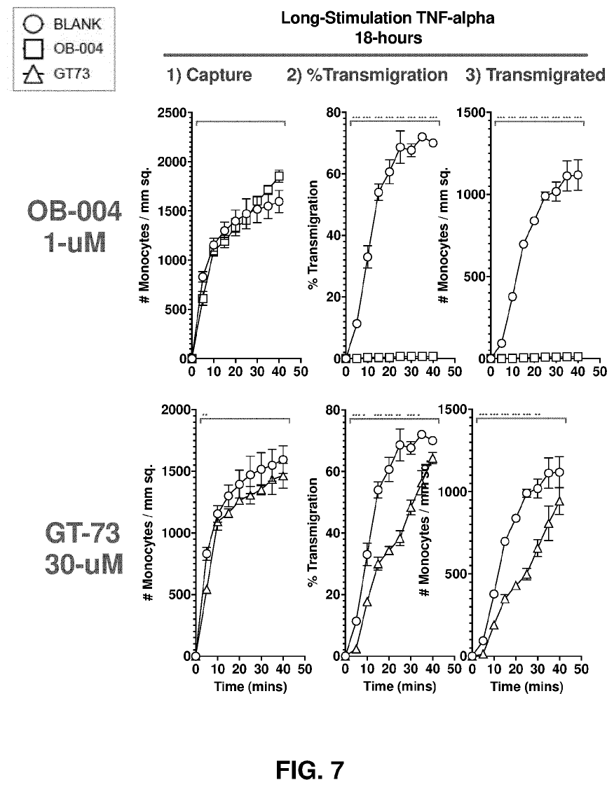

FIG. 7 illustrates the number of monocytes, from Donor 1, captured; % transmigrated and transmigrated on acute activated HUVECS after 18 hours stimulation with 1000U TNF-alpha in the presence of 1P2-CCL2 (Met64Nle OB-004) and GT-73. Monocytes were captured in the presence of 1P2-CCL2 Met64Nle (OB-004) and GT-73, however, 1P2-CCL2 (Met64Nle OB-004) caused a blockade of % transmigration and transmigration.

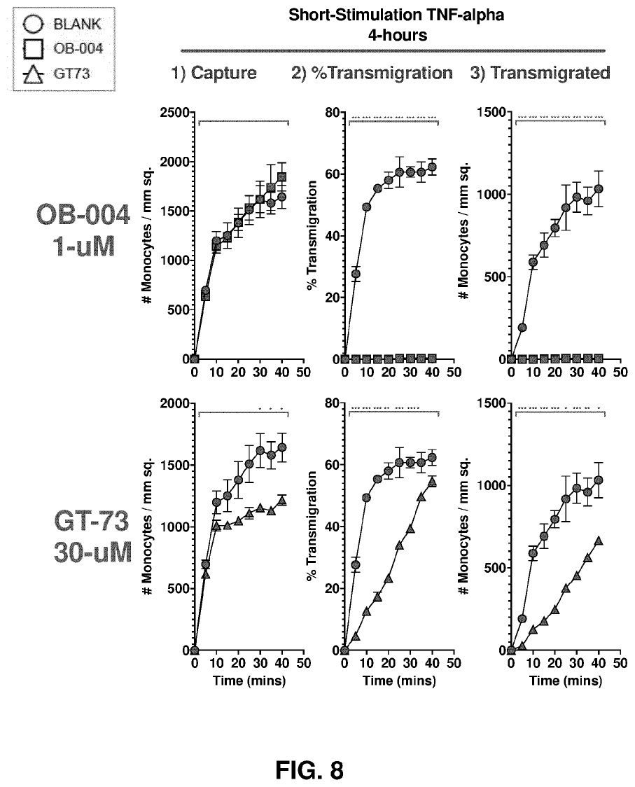

FIG. 8 illustrates the number of monocytes, from Donor 2, captured; % transmigrated and transmigrated on acute activated HUVECS after 4 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

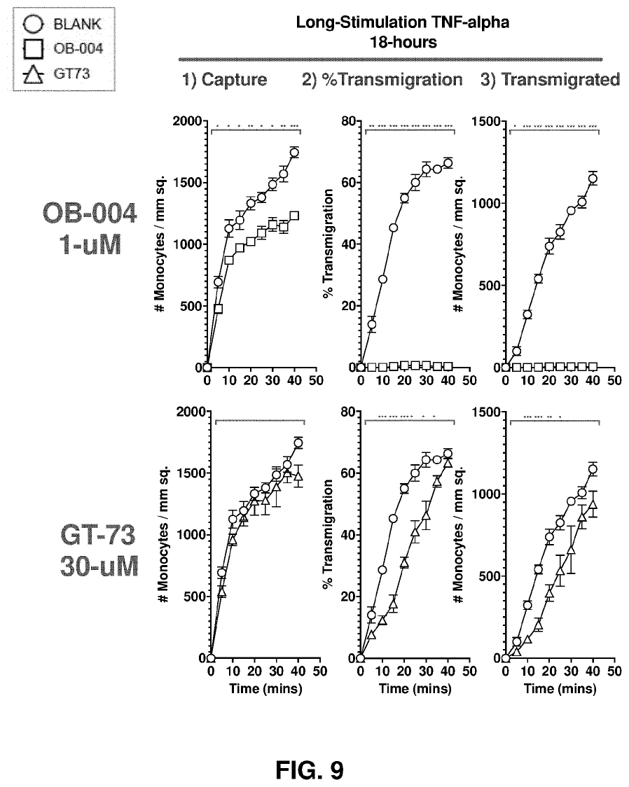

FIG. 9 illustrates the number of monocytes, from Donor 2, captured; % transmigrated and transmigrated on acute activated HUVECS after 18 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

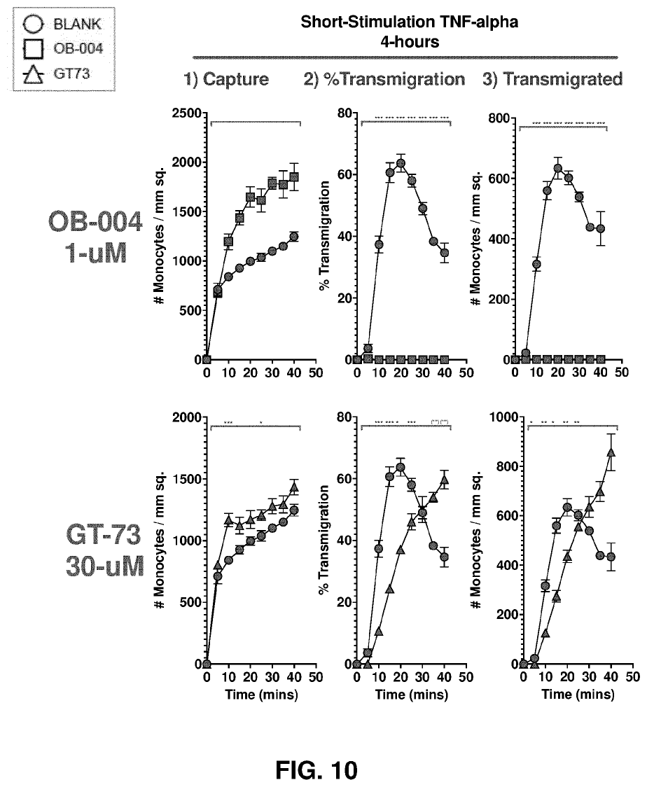

FIG. 10 illustrates the number of monocytes, from Donor 3, captured; % transmigrated and transmigrated on acute activated HUVECS after 4 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

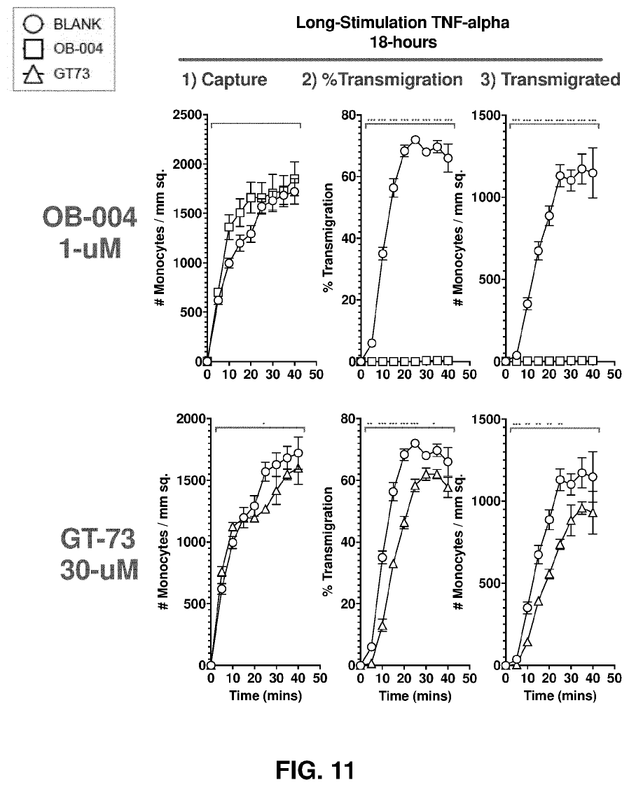

FIG. 11 illustrates the number of monocytes, from Donor 3, captured; % transmigrated and transmigrated on acute activated HUVECS after 18 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

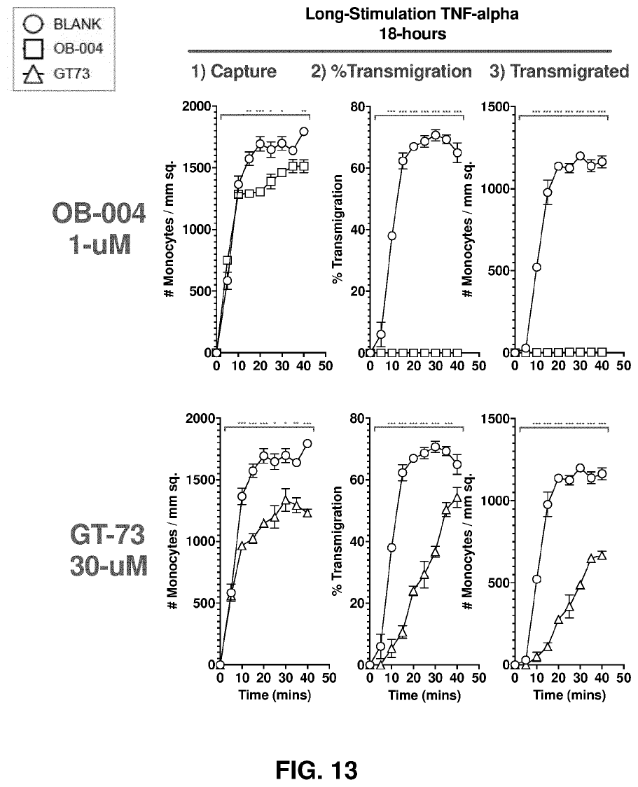

FIG. 13 illustrates the number of monocytes, from Donor 4, captured; % transmigrated and transmigrated on acute activated HUVECS after 4 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

FIG. 13 illustrates the number of monocytes, from Donor 4, captured; % transmigrated and transmigrated on acute activated HUVECS after 18 hours stimulation with 1000U TNF-alpha in the presence of 1P2-CCL2 (Met64Nle OB-004) and GT-73. Monocytes were captured in the presence of 1P2-CCL2 (Met64Nle OB-004) and GT-73, however, 1P2-CCL2 (Met64Nle OB-004) caused a blockade of % transmigration and transmigration.

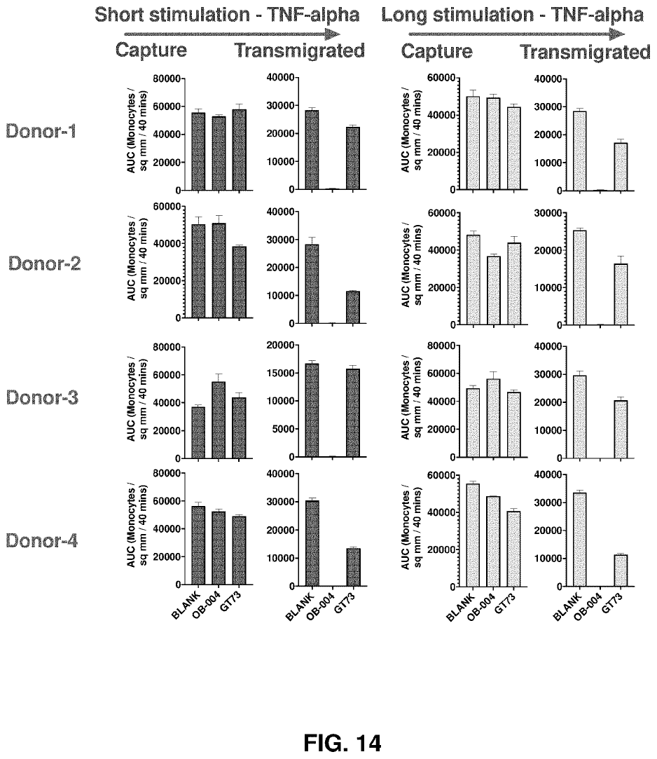

FIG. 14 illustrates an AUC summary of the data shown in FIGS. 6-13 for Donors 1-4.

Blockade of transmission is clearly evidenced in the presence of 1P2-CCL2 (Met64Nle OB-004).

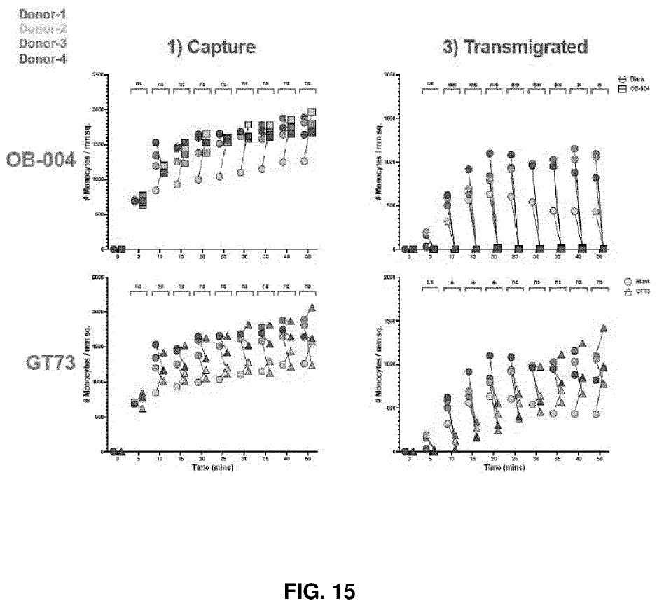

FIG. 15 illustrates the number of monocytes, from Donors 1-4, captured and transmigrated on acute activated HUVECS after 4 hours stimulation with 1000U TNF-alpha in the presence of 1P2-CCL2 (Met64Nle OB-004) and GT-73. The results are presented as a paired analysis of timepoints. Monocytes were captured in the presence of 1P2-CCL2 (Met64Nle OB-004) and GT-73, however, 1P2-CCL2 (Met64Nle OB-004) caused a blockade of transmigration.

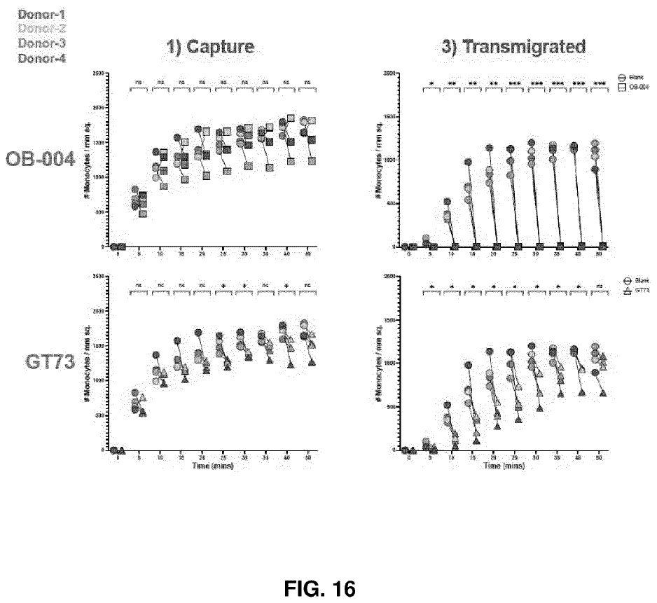

FIG. 16 illustrates the number of monocytes, from Donors 1-4, captured and transmigrated on acute activated HUVECS after 18 hours stimulation with 1000U TNF-alpha in the presence of 1P2-CCL2 (Met64Nle OB-004) and GT-73. The results are presented as a paired analysis of timepoints. Monocytes were captured in the presence of 1P2-CCL2 (Met64Nle OB-004) and GT-73, however, 1P2-CCL2 (Met64Nle OB-004) caused a blockade of transmigration

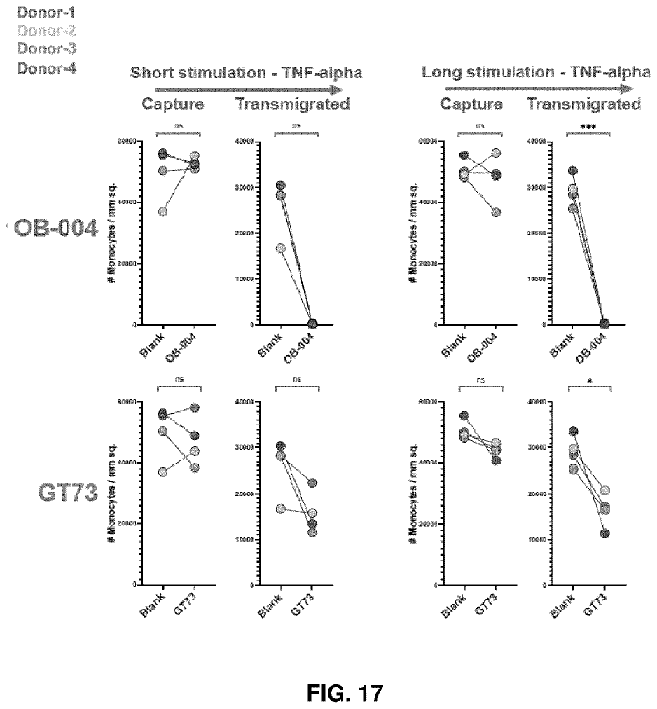

FIG. 17 illustrates the number of monocytes from Donors 1-4, captured and transmigrated on acute activated HUVECS after 4 and 18 hours stimulation with 1000U TNF-alpha in the presence of 1P2-CCL2 (Met64Nle OB-004) and GT-73. The results are presented as a paired analysis of AUC values. Monocytes were captured in the presence of 1P2-CCL2 (Met64Nle OB-004) and GT-73, however, 1P2-CCL2 (Met64Nle OB-004) caused a blockade of transmigration.

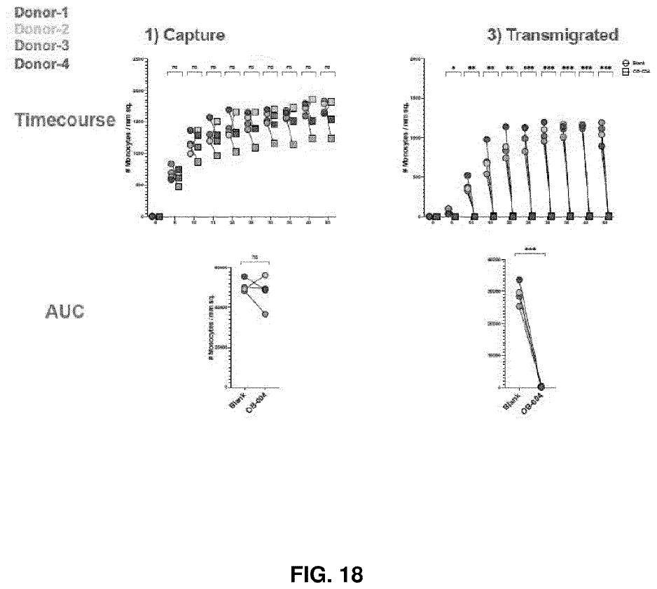

FIG. 18 illustrates 1P2-CCL2 (1P2-CCL2 (Met64Nle OB-004) potently blocks monocyte transmigration in HUVECS activated with TNF-alpha for 18 hours. Results are presented as both a time course and as AUC. Blockade with 1P2-CCL2 (1P2-CCL2 (Met64Nle OB-004) was highly effective, approx. 99% at 1 uM.

A number of peptides, including 1P2, were synthesized and identified as providing CCR2-inhibitory activity, by the methods described above, when provided as the N-terminal portion in a CCL2 variant polypeptide according to the present invention. The peptides identified are detailed in Table 3 below, indicating the measured IC₅₀ of CCR2 inhibition for each peptide.

TABLE 3

| | mean | | | | | | | | | | | | | |
|---|---|---|---|---|---|---|---|---|---|---|---|---|---|---|
| | IC50 | | | N-terminal sequence | | | | | | | | | | |
| ID | (nM) | SEM | n | 0 | 1 | 2 | 3 | 4 | 5 | 6 | 7 | 8 | 9 | 10 |
| CCL2-M64Nle- | 3.43 | 0.833 | 19 | | F | T | N | P | T | W | A | P | V | T |
| 1P2 | | | | | | | | | | | | | | |
| (OB-004) | | | | | | | | | | | | | | |
| (SEQ ID NO: 9) | | | | | | | | | | | | | | |
| CCL2-M64Nle- | 2.2 | 0.126 | 2 | | F | P | Nle | D | G | W | A | P | V | T |
| 2P6 | | | | | | | | | | | | | | |
| (SEQ ID | | | | | | | | | | | | | | |
| NO: 10) | | | | | | | | | | | | | | |
| CCL2-M64Nle- | 5.83 | 2.32 | 2 | | R | P | Y | L | W | K | A | P | V | T |
| 2P5 | | | | | | | | | | | | | | |
| (SEQ ID | | | | | | | | | | | | | | |
| NO: 12) | | | | | | | | | | | | | | |
| CCL2-M64Nle- | 9.47 | 0.34 | 2 | | V | W | N | P | T | W | A | P | V | T |
| NGS-MESR-4 | | | | | | | | | | | | | | |
| (SEQ ID | | | | | | | | | | | | | | |
| NO: 13) | | | | | | | | | | | | | | |
| CCL2-M64Nle- | 9.99 | 2.56 | 5 | I | Y | G | V | G | W | N | A | P | V | T |
| 1P14 | | | | | | | | | | | | | | |
| (SEQ ID | | | | | | | | | | | | | | |
| NO: 14) | | | | | | | | | | | | | | |
| CCL2-M64Nle- | 12 | 4.78 | 6 | | A | F | S | I | M | Q | A | P | V | T |
| 1P8 | | | | | | | | | | | | | | |
| (SEQ ID | | | | | | | | | | | | | | |
| NO: 15) | | | | | | | | | | | | | | |
| CCL2-M64Nle- | 14.2 | 5.3 | 5 | W | V | L | S | S | L | N | A | P | V | T |
| 1P5 | | | | | | | | | | | | | | |
| (SEQ ID | | | | | | | | | | | | | | |
| NO: 17) | | | | | | | | | | | | | | |
| CCL2-M64Nle- | 15.3 | 1.15 | 4 | | V | P | D | W | S | Q | A | P | V | T |
| 1P22 | | | | | | | | | | | | | | |
| (SEQ ID | | | | | | | | | | | | | | |
| NO: 18) | | | | | | | | | | | | | | |
| CCL2-M64Nle- | 18.4 | 2.71 | 2 | | T | P | G | Y | L | K | A | P | V | T |
| NGS-MEO-5 | | | | | | | | | | | | | | |
| (SEQ ID | | | | | | | | | | | | | | |
| NO: 19) | | | | | | | | | | | | | | |
| CCL2-M64Nle- | 24.6 | 2.27 | 4 | | V | T | N | P | T | W | A | P | V | T |
| 1P02-F1V | | | | | | | | | | | | | | |
| (SEQ ID | | | | | | | | | | | | | | |
| NO: 20) | | | | | | | | | | | | | | |
| CCL2-M64Nle- | 26.7 | 4.36 | 4 | W | Y | T | V | G | W | N | A | P | V | T |
| 1P16 | | | | | | | | | | | | | | |
| (SEQ ID | | | | | | | | | | | | | | |
| NO: 21) | | | | | | | | | | | | | | |
| CCL2-M64Nle- | 31.2 | 11.8 | 5 | I | S | W | F | T | F | N | A | P | V | T |
| 1P13 | | | | | | | | | | | | | | |
| (SEQ ID | | | | | | | | | | | | | | |
| NO: 22) | | | | | | | | | | | | | | |
| CCL2-M64Nle- | 32.4 | 5.96 | 5 | M | Y | G | L | G | W | N | A | P | V | T |
| 1P19 | | | | | | | | | | | | | | |
| (SEQ ID | | | | | | | | | | | | | | |
| NO: 23) | | | | | | | | | | | | | | |
| CCL2-M64Nle- | 32.8 | 13 | 3 | | W | R | P | H | W | S | A | P | V | T |
| 1P3 | | | | | | | | | | | | | | |
| (SEQ ID | | | | | | | | | | | | | | |
| NO: 25) | | | | | | | | | | | | | | |
| CCL2-M64Nle- | 38.2 | 3.32 | 2 | | Y | H | W | Q | P | T | A | P | V | T |
| NGS-MESR-3 | | | | | | | | | | | | | | |
| (SEQ ID | | | | | | | | | | | | | | |
| NO: 26) | | | | | | | | | | | | | | |
| CCL2-M64Nle- | 40.2 | 15 | 5 | | W | R | Q | H | N | G | A | P | V | T |
| 1P12 | | | | | | | | | | | | | | |
| (SEQ ID | | | | | | | | | | | | | | |
| NO: 27) | | | | | | | | | | | | | | |
| CCL2-M64Nle- | 41.5 | 2.36 | 2 | | A | W | G | G | I | R | A | P | V | T |
| NGS-MESR-5 | | | | | | | | | | | | | | |
| (SEQ ID | | | | | | | | | | | | | | |
| NO: 28) | | | | | | | | | | | | | | |
| CCL2-M64Nle- | 46.7 | 4.87 | 2 | | W | S | G | Nle | G | K | A | P | V | T |
| 2P2 | | | | | | | | | | | | | | |
| (SEQ ID | | | | | | | | | | | | | | |
| NO: 29) | | | | | | | | | | | | | | |
| CCL2-M64Nle- | 50.2 | 3.96 | 2 | | S | P | S | Y | T | R | A | P | V | T |
| 2P7 | | | | | | | | | | | | | | |
| (SEQ ID | | | | | | | | | | | | | | |
| NO: 31) | | | | | | | | | | | | | | |
| CCL2-M64Nle- | 89.3 | 28.4 | 2 | | — | — | A | P | Q | W | A | A | A | T |
| 2P1 | | | | | | | | | | | | | | |
| (SEQ ID | | | | | | | | | | | | | | |
| NO: 32) | | | | | | | | | | | | | | |
| CCL2-M64Nle- | 117 | 14.8 | 4 | | V | P | S | W | S | Q | A | P | V | T |
| 1P23 | | | | | | | | | | | | | | |
| (SEQ ID | | | | | | | | | | | | | | |
| NO: 33) | | | | | | | | | | | | | | |
| CCL2-M64Nle- | 155 | NA | 1 | | S | Y | S | W | V | R | A | P | V | T |
| NGS-MESR-1 | | | | | | | | | | | | | | |
| (SEQ ID | | | | | | | | | | | | | | |
| NO: 34) | | | | | | | | | | | | | | |
| CCL2-M64Nle- | 167 | 21.5 | 2 | | Nle | P | D | W | N | R | A | P | V | T |
| NGS-MEO-1 | | | | | | | | | | | | | | |
| (SEQ ID | | | | | | | | | | | | | | |
| NO: 35) | | | | | | | | | | | | | | |
| CCL2-M64Nle- | 183 | 4.87 | 2 | | T | P | G | H | Nle | K | A | P | V | T |
| 2P4 | | | | | | | | | | | | | | |
| (SEQ ID NO: | | | | | | | | | | | | | | |
| 37) | | | | | | | | | | | | | | |
| CCL2-M64Nle- | 225 | 43.4 | 2 | — | T | P | G | H | Y | R | A | P | V | T |
| 2P3 | | | | | | | | | | | | | | |
| (SEQ ID | | | | | | | | | | | | | | |
| NO: 39) | | | | | | | | | | | | | | |

Inappropriate recruitment of CCR2-expressing immune cells underlies the inflammatory pathologies in numerous diseases and disorders. Furthermore, CCR2-expressing monocytes recruited to the tumor microenvironment (TME) can be immunosuppressive. The CCR2 inhibitors of the present invention can be used to inhibit or block the inappropriate recruitment of inflammatory or immunosuppressive CCR2-expressing cells to sites of disease pathology. For example, blocking the recruitment of inflammatory monocytes to diseased tissues using a CCR2 inhibitor according to the present invention can achieve a reduction in pathology. Indeed, the transmigration and recruitment data presented herein demonstrates that CCR2 inhibitors according to the present invention have extremely potent CCR2 blocking capability, thereby modifying CCR2-mediated cellular behaviors.

### Example 8: Chimeric Inhibitors with Mouse CCL2

Further to the CCL2 variant polypeptides described in Examples 1-8, which comprise a C-terminal portion from human CCL2, chimeric CCL2 variants comprising a C-terminal portion from mouse CCL2 were tested for CCR2-inhibitory activity.

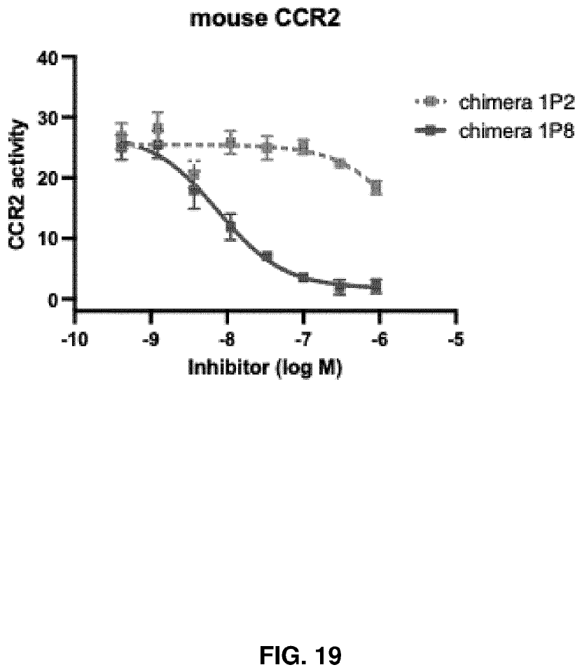

CCL2 variant polypeptides comprising the N-terminal portions from either 1P2 (FTNPTWAPVT, SEQ ID NO: 40) or 1P8 (AFSIMQAPVT, SEQ ID NO: 46) and a C-terminal portion from mouse CCL2 (SEQ ID NO: 72) were produced according to the general methods of Examples 1-4. Chimeric CCL2 variants 1P2 and 1P8 were tested for in vitro CCR2 inhibition as described herein with the exception that mouse CCR2-expressing HEK cells were used to test inhibition of mouse CCR2 activation by agonistic mouse CCL2 (mCCL2, comprising substitution of methionines to norleucines to avoid oxidation during synthesis). As shown in FIG. 19, chimeric 1P2 exhibited an IC₅₀ of 2700 nM and chimeric 1P8 exhibited an IC₅₀ of 7.6 nM.

Chimeric CCL2 variant 1P8 was further tested in an in vivo peritonitis model in male C57Bl/6 mice. Mice were treated with control vehicle (saline) or chimera 1P8 comprising the 1P8 N-terminal portion AFSIMQAPVT (SEQ ID NO: 46) and a C-terminal portion from mouse CCL2 (SEQ ID NO: 72). Chimera 1P8 was administered in saline at different doses (1 mg/kg, 10 mg/kg, 100 mg/kg) by intraperitoneal injection starting 2 days before the induction of peritonitis (by administering 1 ml of thioglycolate 4% injected intraperitoneally) and until euthanasia at 3 different timepoints following induction of peritonitis: 12 h, 24h, 48h (8 mice per dose and timepoint). After euthanasia, peritoneal lavage fluid was obtained by injecting 2 mL of PBS into the peritoneum and then aspirating the exudate to collect immune cells. Peritoneal lavage fluid was centrifuged (450 g, 5 min) and the cell pellet was retained for analysis by flow cytometry, with the leucocyte subpopulation corresponding to infiltrated monocytes/macrophages defined as Ly6G, F4/80*. A viability marker (Viobility™) was used to exclude the dead cells and cellular debris from analysis. In this way, the number of infiltrated monocytes/macrophages was estimated for each mouse for each dose and each timepoint. As shown in FIG. 20, administration of chimeric CCL2 variant 1P8 effectively reduced or blocked monocyte recruitment to the peritoneal cavity in a dose-dependent manner.

CCR2 continues to be actively investigated as a potential drug target for many diseases, ranging from autoimmune diseases, diabetes and chronic pain syndromes, to atherosclerosis, HIV and cancer (Fei et al., Front. Immunol., Nov. 3, 2021). The extremely potent CCR2 blocking capability exhibited by CCR2 inhibitors according to the present invention therefore identify promising candidate molecules for the treatment of diseases or disorders associated with CCR2 signaling including posttraumatic neuralgia, neuropathic pain, inflammatory diseases, chronic obstructive pulmonary disease, diabetic polyneuropathy, cancer (including colorectal cancer, pancreatic cancer, pancreatic ductal adenocarcinoma, liver cancer, non-small cell lung cancer), diabetic nephropathy, diabetes, HIV infection, non-alcoholic steatohepatitis, liver fibrosis, liver cirrhosis, non-alcoholic fatty liver disease, or primary sclerosing cholangitis.

### Example 9: CCR2 Inhibitory Potency Assay

Further CCL2 variants were synthesized according to the method as described in Examples 1-4, and were identified as providing CCR2-inhibitory activity by the methods described above. These further variants were based on the N-terminal portion 1P2 (SEQ ID NO: 40) or 2P6 (SEQ ID NO: 41) but with substitutions at positions 7-10, and were conjugated to the C-terminal fragment of human CCL2 [11-76 human CCL2], in which the methionine residue at position 64 relative to SEQ ID NO: 1) was replaced by norleucine (Nle). The peptides identified are detailed in Table 4 below, indicating the measured IC₅₀ of CCR2 inhibition for each peptide.

TABLE 4

| | | | | | | | | | | | mean | | |
|---|---|---|---|---|---|---|---|---|---|---|---|---|---|
| | N-terminal sequence | | | | | | | | | | IC50 | | |
| ID | 1 | 2 | 3 | 4 | 5 | 6 | 7 | 8 | 9 | 10 | (nM) | SEM | n |
| 1P2-CCL2 | F | T | N | P | T | W | A | P | V | T | 0.806 | 0.0688 | 8 |
| (SEQ ID NO: 40) | | | | | | | | | | | | | |
| 3P1-CCL2 | F | T | N | P | T | W | D | A | F | T | 0.655 | 0.0012 | 2 |
| (SEQ ID NO: 76) | | | | | | | | | | | | | |
| 3P2-CCL2 | F | T | N | P | T | W | D | A | Q | V | 0.883 | 0.0055 | 2 |
| (SEQ ID NO: 77) | | | | | | | | | | | | | |
| 3P3-CCL2 | F | T | N | P | T | W | D | T | G | V | 0.970 | 0.1561 | 2 |
| (SEQ ID NO: 78) | | | | | | | | | | | | | |
| 3P4-CCL2 | F | T | N | P | T | W | D | T | Q | V | 0.646 | 0.0654 | 2 |
| (SEQ ID NO: 79) | | | | | | | | | | | | | |
| 3P5-CCL2 | F | T | N | P | T | W | R | P | S | T | 2.860 | NA | 1 |
| (SEQ ID NO: 80) | | | | | | | | | | | | | |
| 3P6-CCL2 | F | T | N | P | T | W | S | G | V | T | 1.270 | 0.0010 | 2 |
| (SEQ ID NO: 81) | | | | | | | | | | | | | |
| 3P7-CCL2 | F | T | N | P | T | W | A | T | H | V | 1.470 | 0.3625 | 1 |
| (SEQ ID NO: 82) | | | | | | | | | | | | | |
| 3P8-CCL2 | F | T | N | P | T | W | A | P | L | Q | 0.892 | 0.0954 | 2 |
| (SEQ ID NO: 83) | | | | | | | | | | | | | |
| 3P9-CCL2 | F | T | N | P | T | W | D | S | F | S | 0.585 | 0.0686 | 2 |
| (SEQ ID NO: 84) | | | | | | | | | | | | | |
| 3P10-CCL2 | F | T | N | P | T | W | D | G | F | T | 0.676 | 0.1471 | 2 |
| (SEQ ID NO: 85) | | | | | | | | | | | | | |
| 3P11-CCL2 | F | T | N | P | T | W | D | Q | F | T | 0.703 | 0.0099 | 2 |
| (SEQ ID NO: 86) | | | | | | | | | | | | | |
| 3P12-CCL2 | F | T | N | P | T | W | D | R | F | T | 0.625 | 0.2918 | 2 |
| (SEQ ID NO: 87) | | | | | | | | | | | | | |
| 3P13-CCL2 | F | T | N | P | T | W | D | H | F | V | 0.437 | 0.0206 | 2 |
| (SEQ ID NO: 88) | | | | | | | | | | | | | |
| 3P14-CCL2 | F | T | N | P | T | W | D | A | V | T | 0.594 | 0.2129 | 2 |
| (SEQ ID NO: 89) | | | | | | | | | | | | | |
| 3P15-CCL2 | F | T | N | P | T | W | K | P | S | T | 1.530 | 0.1250 | 2 |
| (SEQ ID NO: 90) | | | | | | | | | | | | | |
| 3P16-CCL2 | F | T | N | P | T | W | K | P | Y | A | 1.230 | 0.1110 | 2 |
| (SEQ ID NO: 91) | | | | | | | | | | | | | |
| 3P17-CCL2 | F | T | N | P | T | W | Q | E | Y | S | 1.000 | 0.0755 | 2 |
| (SEQ ID NO: 92) | | | | | | | | | | | | | |
| 3P18-CCL2 | F | T | N | P | T | W | S | P | F | Q | 0.799 | 0.1040 | 2 |
| (SEQ ID NO: 93) | | | | | | | | | | | | | |
| 3P19-CCL2 | F | T | N | P | T | W | S | Q | G | V | 0.914 | 4.3721 | 2 |
| (SEQ ID NO: 94) | | | | | | | | | | | | | |
| 3P20-CCL2 | F | T | N | P | T | W | D | P | F | T | 0.718 | 0.1507 | 2 |
| (SEQ ID NO: 95) | | | | | | | | | | | | | |
| 3P21-CCL2 | F | T | N | P | T | W | D | T | G | T | 1.280 | 0.1730 | 2 |
| (SEQ ID NO: 96) | | | | | | | | | | | | | |
| 3P22-CCL2 | F | T | N | P | T | W | D | A | G | V | 1.000 | 0.1001 | 2 |
| (SEQ ID NO: 97) | | | | | | | | | | | | | |
| 3P23-CCL2 | F | T | N | P | T | W | D | S | V | T | 0.678 | 0.0342 | 2 |
| (SEQ ID NO: 98) | | | | | | | | | | | | | |
| 3P24-CCL2 | F | T | N | P | T | W | D | P | G | V | 0.805 | 0.0304 | 2 |
| (SEQ ID NO: 99) | | | | | | | | | | | | | |
| 4P1-CCL2 | F | P | Nle | D | G | W | R | P | R | V | 8.375 | 3.0050 | 2 |
| (SEQ ID NO: 100) | | | | | | | | | | | | | |
| 4P2-CCL2 | F | P | Nle | D | G | W | G | S | R | V | 2.719 | 0.6470 | 2 |
| (SEQ ID NO: 102) | | | | | | | | | | | | | |
| 4P3-CCL2 | F | P | Nle | D | G | W | H | G | L | T | 1.785 | 0.2115 | 2 |
| (SEQ ID NO: 104) | | | | | | | | | | | | | |
| 4P4-CCL2 | F | P | Nle | D | G | W | R | G | L | T | 2.378 | 0.1950 | 2 |
| (SEQ ID NO: 106) | | | | | | | | | | | | | |
| 4P5-CCL2 | F | P | Nle | D | G | W | R | P | R | Q | 65.790 | NA | 1 |
| (SEQ ID NO: 108) | | | | | | | | | | | | | |
| 4P6-CCL2 | F | P | Nle | D | G | W | R | G | E | T | 1.451 | 0.0920 | 2 |
| (SEQ ID NO: 110) | | | | | | | | | | | | | |
| 4P7-CCL2 | F | P | Nle | D | G | W | G | G | T | V | 2.191 | 0.2400 | 2 |
| (SEQ ID NO: 112) | | | | | | | | | | | | | |
| 4P8-CCL2 | F | P | Nle | D | G | W | G | S | L | T | 1.232 | 0.0015 | 2 |
| (SEQ ID NO: 114) | | | | | | | | | | | | | |
| 4P9-CCL2 | F | P | Nle | D | G | W | R | G | G | V | 1.648 | 0.1360 | 2 |
| (SEQ ID NO: 116) | | | | | | | | | | | | | |
| 4P10-CCL2 | F | P | Nle | D | G | W | V | P | G\| | V | 1.502 | 0.3030 | 2 |
| (SEQ ID NO: 118) | | | | | | | | | | | | | |
| 4P11-CCL2 | F | P | Nle | D | G | W | R | P | G | V | 1.152 | 0.0115 | 2 |
| (SEQ ID NO: 120) | | | | | | | | | | | | | |
| 4P13-CCL2 | F | P | Nle | D | G | W | Q | G | G | V | 0.534 | 0.0464 | 2 |
| (SEQ ID NO: 122) | | | | | | | | | | | | | |
| 4P14-CCL2 | F | P | Nle | D | G | W | V | S | R | T | 0.965 | 0.1550 | 2 |
| (SEQ ID NO: 124) | | | | | | | | | | | | | |
| 4P15-CCL2 | F | P | Nle | D | G | W | R | E | L | V | 1.998 | 0.6735 | 2 |
| (SEQ ID NO: 126) | | | | | | | | | | | | | |
| 4P16-CCL2 | F | P | Nle | D | G | W | R | G | Q | V | 0.820 | 0.1818 | 2 |
| (SEQ ID NO: 128) | | | | | | | | | | | | | |
| 4P17-CCL2 | F | P | Nle | D | G | W | H | P | R | V | 0.663 | 0.1062 | 2 |
| (SEQ ID NO: 130) | | | | | | | | | | | | | |
| 4P18-CCL2 | F | P | Nle | D | G | W | H | P | L | V | 0.585 | 0.0611 | 2 |
| (SEQ ID NO: 132) | | | | | | | | | | | | | |
| 4P19-CCL2 | F | P | Nle | D | G | W | G | G | R | V | 3.248 | 0.9730 | 2 |
| (SEQ ID NO: 134) | | | | | | | | | | | | | |
| 4P20-CCL2 | F | P | Nle | D | G | W | R | G | L | V | 0.930 | 0.2671 | 2 |
| (SEQ ID NO: 136) | | | | | | | | | | | | | |
| 4P21-CCL2 | F | P | Nle | D | G | W | R | G | R | T | 2.117 | 0.1695 | 2 |
| (SEQ ID NO: 138) | | | | | | | | | | | | | |
| 4P22-CCL2 | F | P | Nle | D | G | W | R | P | L | V | 0.759 | 0.0869 | 2 |
| (SEQ ID NO: 140 | | | | | | | | | | | | | |
| 4P23-CCL2 | F | P | Nle | D | G | W | R | P | R | T | 1.020 | 0.0440 | 2 |
| (SEQ ID NO: 142) | | | | | | | | | | | | | |
| 4P24-CCL2 | F | P | Nle | D | G | W | G | P | G | V | 1.647 | 0.4330 | 2 |
| (SEQ ID NO: 144) | | | | | | | | | | | | | |

### Example 10: Evaluation of CCR2 Inhibitor in Bleomycin-Induced Lung Fibrosis in Mice

An exemplary chimeric CCR2 inhibitor comprising the N-terminal portions from 1P8 (AFSIMQAPVT, SEQ ID NO: 46) and a C-terminal portion from mouse CCL2 (SEQ ID NO: 72) as described Example 8 was tested for its effects on the pathology of a mouse model of bleomycin-induced lung fibrosis. Six week old male C567BL/6J mice were sourced from Charles River and used at between 11 and 14 weeks of age at the start of the study.

Administration of bleomycin sulfate (MedChemExpress) was at 1 U/kg (20 mg/mL at 35 uL per mouse) by intratracheal administration. Mice were administered a control vehicle or the CCR2 inhibitor at Day −2 of the study (intraperitoneal injection, twice per day), and a control or bleomycin were administered 5 days per week starting at Day 0 of the study. The mice were euthanized for analysis at Day 21 of the study.
Group 1: bleomycin control (sham).
Group 2: bleomycin+control vehicle.
Group 3: bleomycin+CCR2 inhibitor at 100 mg/kg.
Group 4: bleomycin+CCR2 inhibitor at 33 mg/kg.

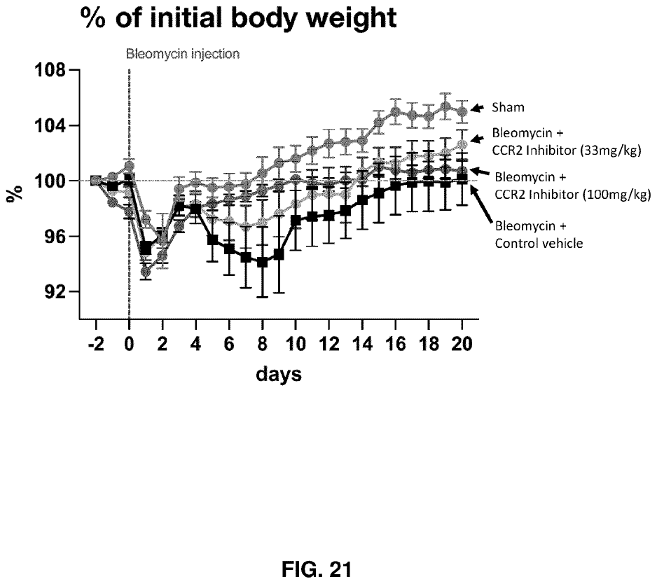

Mice were monitored for body weight during the course of the study. As shown in FIG. 21, mice administered bleomycin lost a greater percentage of body weight compared to sham mice. Weight loss from bleomycin treatment was reduced in mice co-administered the CCR2 inhibitor.

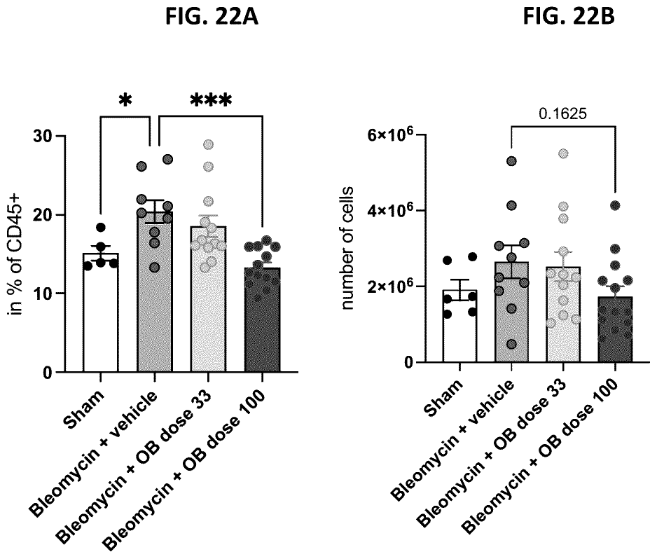

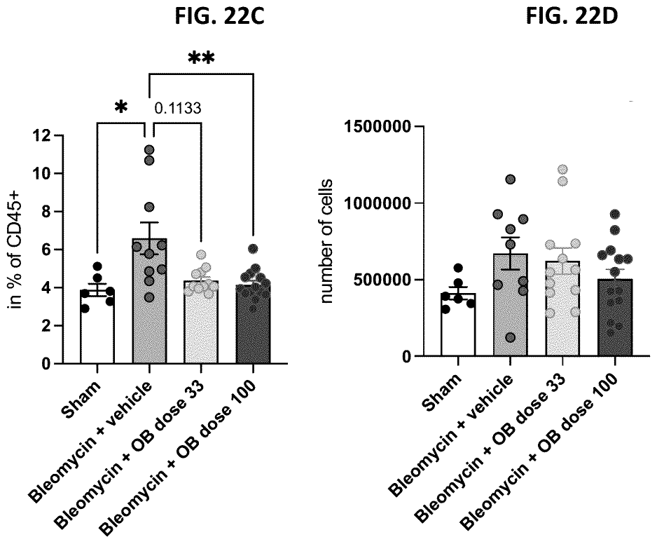

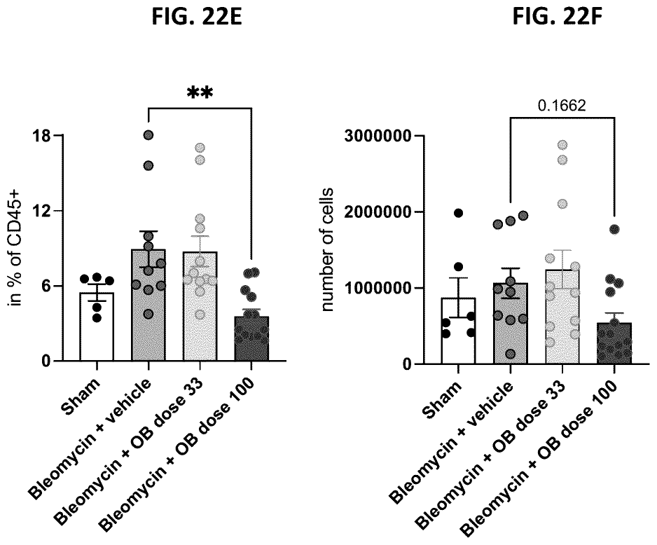

At Day 21 of the study, the infiltrating cells into the lungs were analyzed by flow cytometry. As shown in FIG. 22, co-treatment with the CCR2 inhibitor reduced infiltrating total CD64+ macrophages (FIG. 22A-B), reduced infiltrating CD64+Ly6C−/low SiglecF− interstitial macrophages (FIG. 22C-D), and reduced infiltrating monocyte-derived CD64+ Ly6C+ SiglecF− macrophages (FIG. 22E-F).

The foregoing descriptions of specific embodiments of the present invention have been presented for purposes of illustration and description. They are not intended to be exhaustive or to limit the invention and method of use to the precise forms disclosed. Many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

1. A polypeptide comprising an N-terminal portion and a C-terminal portion, wherein the N-terminal portion comprises an amino acid sequence with 0, 1, 2, 3, or 4 amino acid substitutions relative to any one of SEQ ID NO: 40-70 and 76-145, and wherein the C-terminal portion comprises an amino acid sequence at least 70% identical to SEQ ID NO: 71, 72, 74, or 75.

2. (canceled)

3. The polypeptide of claim 1, wherein the N-terminal portion comprises the amino acid sequence of any one of SEQ ID NO: 40-70 and 76-145.

4. The polypeptide of claim 1, wherein the N-terminal portion comprises the amino acid sequence FTNPTWXXXX (SEQ ID NO: 146), wherein X is any amino acid.

5. The polypeptide of claim 4, wherein the N-terminal portion comprises the amino acid sequence FTNPTW[A or D or R or S or K or Q][P or A or T or G or S or Q or R or H or E][V or F or Q or G or S or H or L or Y][T or V or Q or S or A] (SEQ ID NO: 147).

6-10. (canceled)

11. The polypeptide of claim 1, wherein the C-terminal portion comprises the amino acid sequence of SEQ ID NO: 71, 72, 74, or 75.

12. The polypeptide of claim 1, wherein the polypeptide inhibits CCR2 with an IC50 of less than 300 nM, less than 200 nM, less than 100 nM, less than 50 nM, less than 30 nM, less than 10 nM, or less than 3 nM.

13. A peptide comprising an amino acid sequence with 0, 1, 2, 3, or 4 amino acid substitutions relative to any one of SEQ ID NO: 40-70 and 76-145.

14. (canceled)

15. The peptide of claim 13, comprising the amino acid sequence of any one of SEQ ID NO: 40-70 and 76-145.

16. The peptide of claim 13, comprising the amino acid sequence FTNPTWXXXX (SEQ ID NO: 146), wherein X is any amino acid.

17-21. (canceled)

22. A nucleic acid molecule encoding the polypeptide of claim 1.

23. A vector comprising the nucleic acid molecule of claim 22.

24. A host cell comprising the nucleic acid molecule of claim 22.

25. A pharmaceutical composition comprising the polypeptide of claim 1, and a pharmaceutically acceptable carrier, excipient, and/or stabilizer.

26. The pharmaceutical composition of claim 25, further comprising a therapeutic agent.

27-32. (canceled)

33. A method of inhibiting CCR2 signaling in a cell, comprising contacting the cell with the polypeptide of claim 1.

34. The method of claim 33, wherein the cell is a dendritic cell, a monocyte, a plasma cell, a macrophage, a Kupffer cell, a Langerhans cell, a T cell, a B cell, an erythroid cell, a hepatic stellate cell, a cholangiocyte, a type 2 alveolar cell, a gastric mucus-secreting cell, an NK cell, a hepatocyte, a Hofbauer cell, a spermatid, a fibroblast, a myeloid-derived suppressor cell, a neutrophil, an osteoclast, a stem cell, a basal keratinocyte, a cardiomyocyte, an endothelial cell, a breast glandular cell, a breast myoepithelial cell, a glandular cell, a luminal cell, a theca cell, a spermatogonium, a cytotrophoblast, a smooth muscle cell, an adipocyte, a CCR2-expressing immortalized cell, or a cancer cell.

35. The method of claim 34, wherein the cell is in vitro.

36. The method of claim 34, wherein the cell is in a subject.

37. A method of treating or preventing a disease or disorder associated with CCR2 signaling in a subject, comprising administering to the subject the polypeptide of claim 1.

38. The method of claim 37, wherein the disease or disorder is a pulmonary disease, a cancer, an inflammatory or immune-related disease, a cardiovascular disease, a neurologic disease, a fibrotic condition, a wound or tissue injury, or an infectious disease.

39-44. (canceled)


SEQUENCE LISTING (149 sequences)

SEQ ID NO: 1 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 2 - protein, 70 aa, synthetic construct
APVTCCYNFTNRKISVQRLASYRRITSSKCPKEAVIFKTIVAKEICADPKQKWVQDSMDHLDKQTQTPKT

SEQ ID NO: 3 - protein, 70 aa, synthetic construct
  SITE 58: note = Norleucine
APVTCCYNFTNRKISVQRLASYRRITSSKCPKEAVIFKTIVAKEICADPKQKWVQDSXDHLDKQTQTPKT

SEQ ID NO: 4 - protein, 64 aa, synthetic construct
YNFTNRKISVQRLASYRRITSSKCPKEAVIFKTIVAKEICADPKQKWVQDSMDHLDKQTQTPKT

SEQ ID NO: 5 - protein, 64 aa, synthetic construct
  SITE 52: note = Norleucine
YNFTNRKISVQRLASYRRITSSKCPKEAVIFKTIVAKEICADPKQKWVQDSXDHLDKQTQTPKT

SEQ ID NO: 6 - protein, 76 aa, synthetic construct
FTNPTWAPVTCCYNFTNRKISVQRLASYRRITSSKCPKEAVIFKTIVAKEICADPKQKWVQDSMDHLDKQTQTPKT

SEQ ID NO: 7 - protein, 76 aa, synthetic construct
  SITE 64: note = Norleucine
FTNPTWAPVTCCYNFTNRKISVQRLASYRRITSSKCPKEAVIFKTIVAKEICADPKQKWVQDSXDHLDKQTQTPKT

SEQ ID NO: 8 - protein, 12 aa, synthetic construct
FTNPTWAPVTCC

SEQ ID NO: 9 - protein, 6 aa, synthetic construct
FTNPTW

SEQ ID NO: 10 - protein, 6 aa, synthetic construct
  SITE 3: note = Norleucine
FPXDGW

SEQ ID NO: 11 - protein, 6 aa, synthetic construct
FPMDGW

SEQ ID NO: 12 - protein, 6 aa, synthetic construct
RPYLWK

SEQ ID NO: 13 - protein, 6 aa, synthetic construct
VWNPTW

SEQ ID NO: 14 - protein, 7 aa, synthetic construct
IYGVGWN

SEQ ID NO: 15 - protein, 6 aa, synthetic construct
AFSIMQ

SEQ ID NO: 16 - protein, 6 aa, synthetic construct
  SITE 5: note = Norleucine
AFSIXQ

SEQ ID NO: 17 - protein, 7 aa, synthetic construct
WVLSSLN

SEQ ID NO: 18 - protein, 6 aa, synthetic construct
VPDWSQ

SEQ ID NO: 19 - protein, 6 aa, synthetic construct
TPGYLK

SEQ ID NO: 20 - protein, 6 aa, synthetic construct
VTNPTW

SEQ ID NO: 21 - protein, 7 aa, synthetic construct
WYTVGWN

SEQ ID NO: 22 - protein, 7 aa, synthetic construct
ISWFTFN

SEQ ID NO: 23 - protein, 7 aa, synthetic construct
MYGLGWN

SEQ ID NO: 24 - protein, 7 aa, synthetic construct
  SITE 1: note = Norleucine
XYGLGWN

SEQ ID NO: 25 - protein, 6 aa, synthetic construct
WRPHWS

SEQ ID NO: 26 - protein, 6 aa, synthetic construct
YHWQPT

SEQ ID NO: 27 - protein, 6 aa, synthetic construct
WRQHNG

SEQ ID NO: 28 - protein, 6 aa, synthetic construct
AWGGIR

SEQ ID NO: 29 - protein, 6 aa, synthetic construct
  SITE 4: note = Norleucine
WSGXGK

SEQ ID NO: 30 - protein, 6 aa, synthetic construct
WSGMGK

SEQ ID NO: 31 - protein, 6 aa, synthetic construct
SPSYTR

SEQ ID NO: 32 - protein, 4 aa, synthetic construct
APQW

SEQ ID NO: 33 - protein, 6 aa, synthetic construct
VPSWSQ

SEQ ID NO: 34 - protein, 6 aa, synthetic construct
SYSWVR

SEQ ID NO: 35 - protein, 6 aa, synthetic construct
  SITE 1: note = Norleucine
XPDWNR

SEQ ID NO: 36 - protein, 6 aa, synthetic construct
MPDWNR

SEQ ID NO: 37 - protein, 6 aa, synthetic construct
  SITE 5: note = Norleucine
TPGHXK

SEQ ID NO: 38 - protein, 6 aa, synthetic construct
TPGHMK

SEQ ID NO: 39 - protein, 6 aa, synthetic construct
TPGHYR

SEQ ID NO: 40 - protein, 10 aa, synthetic construct
FTNPTWAPVT

SEQ ID NO: 41 - protein, 10 aa, synthetic construct
  SITE 3: note = Norleucine
FPXDGWAPVT

SEQ ID NO: 42 - protein, 10 aa, synthetic construct
FPMDGWAPVT

SEQ ID NO: 43 - protein, 10 aa, synthetic construct
RPYLWKAPVT

SEQ ID NO: 44 - protein, 10 aa, synthetic construct
VWNPTWAPVT

SEQ ID NO: 45 - protein, 11 aa, synthetic construct
IYGVGWNAPVT

SEQ ID NO: 46 - protein, 10 aa, synthetic construct
AFSIMQAPVT

SEQ ID NO: 47 - protein, 10 aa, synthetic construct
  SITE 5: note = Norleucine
AFSIXQAPVT

SEQ ID NO: 48 - protein, 11 aa, synthetic construct
WVLSSLNAPVT

SEQ ID NO: 49 - protein, 10 aa, synthetic construct
VPDWSQAPVT

SEQ ID NO: 50 - protein, 10 aa, synthetic construct
TPGYLKAPVT

SEQ ID NO: 51 - protein, 10 aa, synthetic construct
VTNPTWAPVT

SEQ ID NO: 52 - protein, 11 aa, synthetic construct
WYTVGWNAPVT

SEQ ID NO: 53 - protein, 11 aa, synthetic construct
ISWFTFNAPVT

SEQ ID NO: 54 - protein, 11 aa, synthetic construct
MYGLGWNAPVT

SEQ ID NO: 55 - protein, 11 aa, synthetic construct
  SITE 1: note = Norleucine
XYGLGWNAPVT

SEQ ID NO: 56 - protein, 10 aa, synthetic construct
WRPHWSAPVT

SEQ ID NO: 57 - protein, 10 aa, synthetic construct
YHWQPTAPVT

SEQ ID NO: 58 - protein, 10 aa, synthetic construct
WRQHNGAPVT

SEQ ID NO: 59 - protein, 10 aa, synthetic construct
AWGGIRAPVT

SEQ ID NO: 60 - protein, 10 aa, synthetic construct
  SITE 4: note = Norleucine
WSGXGKAPVT

SEQ ID NO: 61 - protein, 10 aa, synthetic construct
WSGMGKAPVT

SEQ ID NO: 62 - protein, 10 aa, synthetic construct
SPSYTRAPVT

SEQ ID NO: 63 - protein, 8 aa, synthetic construct
APQWAAAT

SEQ ID NO: 64 - protein, 10 aa, synthetic construct
VPSWSQAPVT

SEQ ID NO: 65 - protein, 10 aa, synthetic construct
SYSWVRAPVT

SEQ ID NO: 66 - protein, 10 aa, synthetic construct
  SITE 1: note = Norleucine
XPDWNRAPVT

SEQ ID NO: 67 - protein, 10 aa, synthetic construct
MPDWNRAPVT

SEQ ID NO: 68 - protein, 10 aa, synthetic construct
  SITE 5: note = Norleucine
TPGHXKAPVT

SEQ ID NO: 69 - protein, 10 aa, synthetic construct
TPGHMKAPVT

SEQ ID NO: 70 - protein, 10 aa, synthetic construct
TPGHYRAPVT

SEQ ID NO: 71 - protein, 63 aa, synthetic construct
  SITE 9: note = Norleucine
  SITE 12: note = Norleucine
  SITE 62: note = Norleucine
CCYSFTSKXIPXSRLESYKRITSSRCPKEAVVFVTKLKREVCADPKKEWVQTYIKNLDRNQXR

SEQ ID NO: 72 - protein, 63 aa, synthetic construct
CCYSFTSKMIPMSRLESYKRITSSRCPKEAVVFVTKLKREVCADPKKEWVQTYIKNLDRNQMR

SEQ ID NO: 73 - protein, 73 aa, Mus musculus
QPDAVNAPLTCCYSFTSKMIPMSRLESYKRITSSRCPKEAVVFVTKLKREVCADPKKEWVQTYIKNLDRNQMR

SEQ ID NO: 74 - protein, 66 aa, synthetic construct
CCYNFTNRKISVQRLASYRRITSSKCPKEAVIFKTIVAKEICADPKQKWVQDSMDHLDKQTQTPKT

SEQ ID NO: 75 - protein, 66 aa, synthetic construct
CCYNFTNRKISVQRLASYRRITSSKCPKEAVIFKTIVAKEICADPKQKWVQDSXDHLDKQTQTPKT

SEQ ID NO: 76 - protein, 10 aa, synthetic construct
FTNPTWDAFT

SEQ ID NO: 77 - protein, 10 aa, synthetic construct
FTNPTWDAQV

SEQ ID NO: 78 - protein, 10 aa, synthetic construct
FTNPTWDTGV

SEQ ID NO: 79 - protein, 10 aa, synthetic construct
FTNPTWDTQV

SEQ ID NO: 80 - protein, 10 aa, synthetic construct
FTNPTWRPST

SEQ ID NO: 81 - protein, 10 aa, synthetic construct
FTNPTWSGVT

SEQ ID NO: 82 - protein, 10 aa, synthetic construct
FTNPTWATHV

SEQ ID NO: 83 - protein, 10 aa, synthetic construct
FTNPTWAPLQ

SEQ ID NO: 84 - protein, 10 aa, synthetic construct
FTNPTWDSFS

SEQ ID NO: 85 - protein, 10 aa, synthetic construct
FTNPTWDGFT

SEQ ID NO: 86 - protein, 10 aa, synthetic construct
FTNPTWDQFT

SEQ ID NO: 87 - protein, 10 aa, synthetic construct
FTNPTWDRFT

SEQ ID NO: 88 - protein, 10 aa, synthetic construct
FTNPTWDHFV

SEQ ID NO: 89 - protein, 10 aa, synthetic construct
FTNPTWDAVT

SEQ ID NO: 90 - protein, 10 aa, synthetic construct
FTNPTWKPST

SEQ ID NO: 91 - protein, 10 aa, synthetic construct
FTNPTWKPYA

SEQ ID NO: 92 - protein, 10 aa, synthetic construct
FTNPTWQEYS

SEQ ID NO: 93 - protein, 10 aa, synthetic construct
FTNPTWSPFQ

SEQ ID NO: 94 - protein, 10 aa, synthetic construct
FTNPTWSQGV

SEQ ID NO: 95 - protein, 10 aa, synthetic construct
FTNPTWDPFT

SEQ ID NO: 96 - protein, 10 aa, synthetic construct
FTNPTWDTGT

SEQ ID NO: 97 - protein, 10 aa, synthetic construct
FTNPTWDAGV

SEQ ID NO: 98 - protein, 10 aa, synthetic construct
FTNPTWDSVT

SEQ ID NO: 99 - protein, 10 aa, synthetic construct
FTNPTWDPGV

SEQ ID NO: 100 - protein, 10 aa, synthetic construct
  SITE 3: note = Norleucine
FPXDGWRPRV

SEQ ID NO: 101 - protein, 10 aa, synthetic construct
FPMDGWRPRV

SEQ ID NO: 102 - protein, 10 aa, synthetic construct
  SITE 3: note = Norleucine
FPXDGWGSRV

SEQ ID NO: 103 - protein, 10 aa, synthetic construct
FPMDGWGSRV

SEQ ID NO: 104 - protein, 10 aa, synthetic construct
  SITE 3: note = Norleucine
FPXDGWHGLT

SEQ ID NO: 105 - protein, 10 aa, synthetic construct
FPMDGWHGLT

SEQ ID NO: 106 - protein, 10 aa, synthetic construct
  SITE 3: note = Norleucine
FPXDGWRGLT

SEQ ID NO: 107 - protein, 10 aa, synthetic construct
FPMDGWRGLT

SEQ ID NO: 108 - protein, 10 aa, synthetic construct
  SITE 3: note = Norleucine
FPXDGWRPRQ

SEQ ID NO: 109 - protein, 10 aa, synthetic construct
FPMDGWRPRQ

SEQ ID NO: 110 - protein, 10 aa, synthetic construct
  SITE 3: note = Norleucine
FPXDGWRGET

SEQ ID NO: 111 - protein, 10 aa, synthetic construct
FPMDGWRGET

SEQ ID NO: 112 - protein, 10 aa, synthetic construct
  SITE 3: note = Norleucine
FPXDGWGGTV

SEQ ID NO: 113 - protein, 10 aa, synthetic construct
FPMDGWGGTV

SEQ ID NO: 114 - protein, 10 aa, synthetic construct
  SITE 3: note = Norleucine
FPXDGWGSLT

SEQ ID NO: 115 - protein, 10 aa, synthetic construct
FPMDGWGSLT

SEQ ID NO: 116 - protein, 10 aa, synthetic construct
  SITE 3: note = Norleucine
FPXDGWRGGV

SEQ ID NO: 117 - protein, 10 aa, synthetic construct
FPMDGWRGGV

SEQ ID NO: 118 - protein, 10 aa, synthetic construct
  SITE 3: note = Norleucine
FPXDGWVPGV

SEQ ID NO: 119 - protein, 10 aa, synthetic construct
FPMDGWVPGV

SEQ ID NO: 120 - protein, 10 aa, synthetic construct
  SITE 3: note = Norleucine
FPXDGWRPGV

SEQ ID NO: 121 - protein, 10 aa, synthetic construct
FPMDGWRPGV

SEQ ID NO: 122 - protein, 10 aa, synthetic construct
  SITE 3: note = Norleucine
FPXDGWQGGV

SEQ ID NO: 123 - protein, 10 aa, synthetic construct
FPMDGWQGGV

SEQ ID NO: 124 - protein, 10 aa, synthetic construct
  SITE 3: note = Norleucine
FPXDGWVSRT

SEQ ID NO: 125 - protein, 10 aa, synthetic construct
FPMDGWVSRT

SEQ ID NO: 126 - protein, 10 aa, synthetic construct
  SITE 3: note = Norleucine
FPXDGWRELV

SEQ ID NO: 127 - protein, 10 aa, synthetic construct
FPMDGWRELV

SEQ ID NO: 128 - protein, 10 aa, synthetic construct
  SITE 3: note = Norleucine
FPXDGWRGQV

SEQ ID NO: 129 - protein, 10 aa, synthetic construct
FPMDGWRGQV

SEQ ID NO: 130 - protein, 10 aa, synthetic construct
  SITE 3: note = Norleucine
FPXDGWHPRV

SEQ ID NO: 131 - protein, 10 aa, synthetic construct
FPMDGWHPRV

SEQ ID NO: 132 - protein, 10 aa, synthetic construct
  SITE 3: note = Norleucine
FPXDGWHPLV

SEQ ID NO: 133 - protein, 10 aa, synthetic construct
FPMDGWHPLV

SEQ ID NO: 134 - protein, 10 aa, synthetic construct
  SITE 3: note = Norleucine
FPXDGWGGRV

SEQ ID NO: 135 - protein, 10 aa, synthetic construct
FPMDGWGGRV

SEQ ID NO: 136 - protein, 10 aa, synthetic construct
  SITE 3: note = Norleucine
FPXDGWRGLV

SEQ ID NO: 137 - protein, 10 aa, synthetic construct
FPMDGWRGLV

SEQ ID NO: 138 - protein, 10 aa, synthetic construct
  SITE 3: note = Norleucine
FPXDGWRGRT

SEQ ID NO: 139 - protein, 10 aa, synthetic construct
FPMDGWRGRT

SEQ ID NO: 140 - protein, 10 aa, synthetic construct
  SITE 3: note = Norleucine
FPXDGWRPLV

SEQ ID NO: 141 - protein, 10 aa, synthetic construct
FPMDGWRPLV

SEQ ID NO: 142 - protein, 10 aa, synthetic construct
  SITE 3: note = Norleucine
FPXDGWRPRT

SEQ ID NO: 143 - protein, 10 aa, synthetic construct
FPMDGWRPRT

SEQ ID NO: 144 - protein, 10 aa, synthetic construct
  SITE 3: note = Norleucine
FPXDGWGPGV

SEQ ID NO: 145 - protein, 10 aa, synthetic construct
FPMDGWGPGV

SEQ ID NO: 146 - protein, 10 aa, synthetic construct
  SITE 7: note = Any amino acid
  SITE 8: note = Any amino acid
  SITE 9: note = Any amino acid
  SITE 10: note = Any amino acid
FTNPTWXXXX

SEQ ID NO: 147 - protein, 10 aa, synthetic construct
  SITE 7: note = A or D or R or S or K or Q
  SITE 8: note = P or A or T or G or S or Q or R or H or E
  SITE 9: note = V or F or Q or G or S or H or L or Y
  SITE 10: note = T or V or Q or S or A
FTNPTWXXXX

SEQ ID NO: 148 - protein, 10 aa, synthetic construct
  SITE 3: note = Norleucine or M
  SITE 7: note = Any amino acid
  SITE 8: note = Any amino acid
  SITE 9: note = Any amino acid
  SITE 10: note = Any amino acid
FPXDGWXXXX

SEQ ID NO: 149 - protein, 10 aa, synthetic construct
  SITE 3: note = Norleucine or M
  SITE 7: note = A or R or G or H or V or Q
  SITE 8: note = P or S or G or E
  SITE 9: note = V or R or L or E or T or G or Q
  SITE 10: note = T or V or Q
FPXDGWXXXX